(19) United States
(12) Patent Application Publication
(10) Pub. No.: US 2022/0211606 A1
(43) Pub. Date: Jul. 7, 2022

(54) LIQUID COMPOSITION FOR INFLUENCING THE MICROBIOTA ON A SUBJECT'S SKIN COMPRISING CHITOSAN

(71) Applicant: Medoderm GmbH, Mainz (DE)

(72) Inventors: Günter Bauer, Schmalfeld (DE); Manfred Beleut, Seukendorf (DE); Karsten Henco, Düsseldorf (DE)

(21) Appl. No.: 17/601,783

(22) PCT Filed: Mar. 30, 2020
(86) PCT No.: PCT/EP2020/058966

§ 371 Date: Oct. 6, 2021

(30) Foreign Application Priority Data
Apr. 8, 2019 (GB) 1904927.9
Apr. 30, 2019 (GB) 1906057.3

(51) Int. Cl. A61K 8/73; A61K 8/365; A61Q 17/00; A61Q 19/00

(52) U.S. Cl. CPC A61K 8/736; A61K 8/365; A61Q 19/005; A61Q 19/007; A61Q 17/005

(57) ABSTRACT

The present invention relates to liquid compositions as well as the use of liquid compositions for differentially promoting the growth of the microbiome. The liquid composition forms a membrane of the invention when applied to the skin of a subject. The liquid composition as well as the membrane comprise chitosan or a salt thereof with a degree of acetylation of 20% or less and at least one further agent. The present invention further relates to pharmaceutical compositions for the treatment of dysbiosis.

### BACKGROUND OF THE INVENTION

The polysaccharide chitosan is the at least partially N-deacetylated derivative of chitin. Chitin can be found widely in the exoskeletons of arthropods, gels, crustaceans and the cuticles of insects. It is usually derived from such natural sources. Chitosan in general is synthetically prepared by hydrolysis of chitin, although it can also be naturally derived directly, e.g. from certain fungi in which it occurs. The different solubilities of chitin and chitosan in dilute acids are commonly used to distinguish between the two polysaccharides. Chitosan, the soluble form, can have a degree of acetylation (DA) between 0% and about 60%, the upper limit depending on parameters such as processing conditions, molecular weight, and solvent characteristics. While soluble in acidic aqueous media, chitosan precipitates at a pH of above 6.3.

Both, chitin and chitosan are promising polymers for biomedical applications because of their biocompatibility, biodegradability and structural similarity to the glycosaminoglycans and lack of immunogenic antigenicity.

For example, WO 2011/026498 A1 discloses a tissue dressing material, which has as the main component deacetylated native chitosan.

WO 2011/026869 A1 discloses a tissue dressing kit comprising a tissue dressing material comprising deacetylated native chitosan for being applied in contact with the tissue of a patient and a detachment solvent for removing the tissue dressing material from the tissue.

Further, chitosan is also known to be useful as a cosmetic agent, e.g. in dental care, hair care, skin care and oral care (for a comprehensive summary, see review of Aranaz et al., “Cosmetics and Cosmeceutical Applications of Chitin, Chitosan and Their Derivatives”, Polymers 2018, 10, 213). For example, chitosan derivatives have been described as antimicrobiants, humectants, antioxidants, skin conditioners, cleansing agents, emollients and skin protecting agents. Further, chitosan has been used as a vehicle for active ingredients in skin care in form of encapsulating nanoparticles.

There are several ways the chitosan could have been present in these cosmetic products. For example, chitosan may be dissolved in a liquid or it can be applied without dissolving the chitosan. When applied in a dissolved form to the skin, the chitosan may or may not precipitate and form a film covering the skin but this may depend on various factors such as the pH or the amount of chitosan of the liquid cosmetic formulation and/or the presence of further cosmetic agents. As was already described above, chitosan usually precipitates at a pH above 6.3 and depends much on the integrity and surface conditions of the skin. However, the natural pH of the skin is between pH 4.0 and 6.0 (Gruber 2016, “Behandlungsstrategien bei stagnierenden Wunden”, pages 1-42). The skin is in general in better condition with pH values of below 5.0 than skin with pH above 5.0 (e.g. for resident skin flora, biophysical parameters of barrier function, moisturization and scaling) and a precipitating film of chitosan having a pH of 6.3 is therefore not ideal for skin care applications (see Lambers et al. “Natural skin surface pH is on average below 5, which is beneficial for its resident flora.”, Int J Cosmet Sci. 2006 October; 28(5):359-70).

The biological integrity of the skin as the largest human organ is of critical importance. Severe burns or injuries need medicinal intervention to restore the skin integrity and assist natural repair mechanisms. However, not only these severe conditions need attention. Even small and local topical disturbances of the skin, which may not necessarily be due to a pathological condition need to be taken care of. These disturbances may e.g. be caused by mechanical stress factors, temperature stress, radiation stress, variation exchange or transmission of external factors such as oxygen, carbon dioxide, metal ions, chemicals, water or nutrients or internal factors or combination thereof. The disturbances may also be caused by an imbalance e.g. of the microflora or microbiome of the skin. Furthermore, disturbances may be caused by skin peeling, laser treatments, plasma treatments, tattoos and removal of tattoos.

Such disturbances may not directly cause medicinal symptoms such as severe lesions or wounds with a direct need of medicinal intervention but may nevertheless be accompanied by surface pain, e.g. at stressed nails, itching, burn wet skin areas, smell, rough cracky fissures, dryness, ugliness and the like.

A long lasting cosmetic membrane, which is preferably almost invisible, supporting the care of such unbalanced skin surface, while lowering or avoiding any of the symptoms described above or any aggravation towards severe skin lesions, would be of great value for cosmetic use. Also, a cosmetic membrane which may serve as stable support for conventional make-up with no disturbing optical impact would be of great value. Furthermore, a liquid composition comprising chitosan for promoting or re-establishing a healthy microbiome would be very useful, for cosmetic and medicinal applications.

It is thus an object of the present invention to provide further chitosan-based cosmetic products for topical skin application with beneficial properties. It is further an object of the present invention to provide chitosan-based products for topical skin application which beneficially influence the microbiome of the skin and/or support the formation of a healthy microbiome on the skin. It is still another object of the present invention to provide chitosan-based pharmaceutical compositions for the re-establishment or promotion of a healthy microbiome.

### SUMMARY

The object can be achieved by the liquid composition as defined herein and use thereof. The object is also achieved by a membrane which forms upon topical application of the liquid composition on the skin of a subject and the use of such membrane. Furthermore, the object can be achieved by a pharmaceutical composition.

Therefore, in a first aspect, the present invention relates to a liquid cosmetic composition comprising chitosan for use in differentially promoting the growth of the microbiota on a subject's ectodermal tissue.

In one embodiment, the liquid cosmetic composition is applied topically to form a cosmetic membrane.

In another embodiment, the formed membrane has a thickness of 0.001 nm to 50 μm, preferably 0.001 nm to 10 μm and more preferably from 0.001 nm to 1 μm.

In another embodiment, the liquid cosmetic composition which forms a membrane differentially promotes the growth of one or more microbial taxa relative to another microbial taxa.

In another embodiment, the liquid cosmetic composition which forms a membrane promotes the growth of one or more beneficial microbial taxa relative to one or more pathogenic microbial taxa.

In another embodiment, the at least one beneficial taxa comprises Staphylococcus epidermidis, Staphylococcus mitis, Staphylococcus capitis, Corynebacterium specs., Propionibacterium acnes, Malassezia pachydermatis, Streptococcus spec., Streptococcus spec., Lactobacillus spec., Micrococcus spec. and Bacillus spec.,

In another embodiment, the at least one pathogenic taxa comprises Staphylococcus aureus, Staphylococcus epidermidis, Pseudomonas aeruginosa, Enterococcus faecalis/faecium, E. coli, Klebsiella pneumoniae, Candida albicans, Microsporum canis, Acinetobacter baumanii, Staphylococcus intermedius and Staphylococcus pseudointermedius.

In another embodiment, the chitosan has a degree of acetylation of 15% or less, more preferably 10% or less, even more preferably 5% or less or even more preferably 2.5% or less.

In another embodiment, the liquid cosmetic composition comprises one further cosmetic agent.

In another embodiment, the one further cosmetic agent is selected from the group comprising urea, glycolic acid, glyoxylic acid, glycerol, pentylene glycol, lactic acid, ascorbic acid, pyroglutamic acid, citric acid, tartaric acid, fumaric acid, succinic acid, malic acid, mandelic acid, aloe vera, rosa gallica, hyaluronic acid, salicylic acid, gallic acid, cellulose and derivatives thereof, pectin and derivatives thereof, gummi arabicum, dextrines, cyclodextrines, xanthan gum, thiocyanate, amino acids, sorbic acid, sodium chloride or combinations thereof.

In another embodiment, the at least one further cosmetic agent comprises glycolic acid and lactic acid.

In another embodiment, the composition comprises

0.01 to 4.0% (w/w) chitosan,

0.005 to 2.5% (w/w) glycolic acid,

0.005 to 2.5% (w/w) lactic acid,

based on the total weight of the liquid cosmetic composition.

In another embodiment, the liquid cosmetic composition comprises a disinfectant.

In another embodiment, the disinfectant comprises an alcohol, aldehyde, iodine, chlorine, quarternary ammonia compound, peroxide, amphotenside, phenol, alkylamine, acid and/or base.

In another embodiment, the ectodermal tissue is the skin.

In another embodiment, the ectodermal tissue is the epidermis.

In another embodiment, the epidermis is damaged epidermis, wherein the damage comprises sunburn, acne, cuts, abrasions, cuts, pimples, blisters, stings, burns, ageing, irritated skin, radiated skin, laser treated skin, plasma treated skin, a tattoo, the removal of a tattoo, skin peeling, scar tissue, dry skin, fatty skin, cracks, stretch marks or wrinkling.

In another embodiment, the subject's ectodermal tissue, skin, epidermis or damaged epidermis has previously been sterilized or disinfected.

In another embodiment, the epidermis is stressed epidermis, wherein the stress is caused by prostheses, endoprostheses, orthoses, exoskeletons, plasters, compression bandages, stockings, bandages, latex protection, massagers, masks for ventilation, apnea prevention, work- or protective clothing, gloves, pacifiers, tight clothes or shoes, disinfectants, cosmetic treatments, cosmetic products, preservatives, cleaning agents, sweat, lack of body hygiene, long lying or sitting, wound dressings, sunburns, burns, stings, radiation, laser treatment, plasma treatment, tattooing and/or removal of tattoos.

In another embodiment, the stress is caused by prostheses, endoprostheses, orthoses, exoskeletons, plasters, compression bandages, stockings, bandages, latex protection, massagers, masks for ventilation, work- or protective clothing, gloves, pacifiers, tight clothes or shoes, long lying or sitting or wound dressings.

In another embodiment, the subject's ectodermal tissue, skin, epidermis, damaged or stressed epidermis has previously been sterilized or disinfected.

In a second aspect, the present invention relates to a liquid cosmetic composition comprising

a) chitosan or a salt thereof, wherein the degree of acetylation of the chitosan is 20% or less, and

b) at least one further cosmetic agent;

wherein the pH of the liquid cosmetic composition is from about 4.0 to about 6.5;

and wherein the at least one further cosmetic agent comprises glycolic and lactic acid.

In one embodiment of the second aspect, the degree of acetylation of the chitosan is 15% or less, more preferably 10% or less, even more preferably 5% or less, or even more preferably 2.5% or less.

In another embodiment of the second aspect, the composition comprises

0.010 to 4.0% (w/w) chitosan,

0.005 to 2.5% (w/w) glycolic acid,

0.005 to 2.5% (w/w) lactic acid,

based on the total weight of the liquid cosmetic composition.

In a third aspect, the present invention relates to a kit comprising a liquid cosmetic composition according to any of the preceding embodiments, further comprising a disinfectant, preferentially selected from the group comprising an alcohol, aldehyde, iodine, chlorine, quarternary ammonia compound, peroxide, amphotenside, phenol, alkylamine, acid and/or base.

### BRIEF DESCRIPTION OF FIGURES

FIG. 1: Change in pH of skin after treatment with cosmetic compositions comprising at least one further cosmetic agent. The figure shows that the presence of a chitosan membrane significantly reduces the pH of washed skin with a pH of above 6.0.

FIG. 2: Storage capacity of membranes formed from four cosmetic liquid compositions A to D including cosmetic compositions comprising at least one further cosmetic agent according to the invention (compositions A and B), evaluated with Skin PAMPA at three different timepoints.

FIG. 3: Photographic result of test formulation E as described in Example 3.

FIG. 4: Photographic result of test formulation F as described in Example 3.

FIG. 5: Photographic result of test formulation G as described in Example 3.

FIG. 6: Photographic result of test formulation E as described in Example 4, after defined time points without contact to surfaces or skin regions.

FIG. 7: Photographic result of test formulation E as described in Example 4, with the samples taken directly after contact to a textile, which had contact to normal skin and after 1 h incubation after the contact.

FIG. 8: Results of test campagne as regards preference and/or recommendation of test formulation E in view of the different applications abrasions, pimples, cracks, insect bites, blisters, scar care and lip tingling.

FIG. 9: Evaluation of testers of test formulation E as regards effects in the treatment or care of abrasions, pimples, cracks, insect bites, blisters, scar care and lip tingling.

### DETAILED DESCRIPTION

Use of a Liquid Cosmetic Composition Comprising Chitosan for Differentially Promoting the Growth of the Microbiome

It was surprisingly found that a liquid cosmetic composition comprising chitosan differentially promotes the growth of the skin's healthy microbiome. For example, the liquid cosmetic composition comprising chitosan and the membrane formed by the liquid cosmetic composition comprising chitosan promote the growth of one beneficial microbial taxa relative to another less beneficial or even pathogenic microbial taxa. This mechanism is helpful for various skin care applications listed further below. This mechanism may be especially advantageous after or simultaneous to disinfection which is frequently used in cosmetical skin applications such as tattoo-applications or skin impurities such as acne.

In the first aspect, the present invention relates to the use of a liquid cosmetic composition comprising chitosan for differentially promoting the growth of the microbiota on a subject's ectodermal tissue.

In one embodiment, the liquid cosmetic composition comprising chitosan differentially promotes the growth of the microbiota on a subject's ectodermal tissue. In one embodiment the liquid cosmetic composition comprising chitosan engrafts or improves the colonization of a microbial taxa on a subject's ectodermal tissue. In another embodiment, the liquid cosmetic composition comprising chitosan modulates a microbial taxa on a subject's ectodermal tissue. In a preferred embodiment the modulating a microbial taxa comprises an increase or decrease in the abundance of the taxa. In another preferred embodiment modulating a microbial taxa comprises an increase or decrease in the abundance of the taxa relative to the abundance of said microbial taxa in the absence of the liquid composition. In another preferred embodiment, the liquid cosmetic composition comprising chitosan modulates the microbial diversity on the subject's ectodermal tissue. In another preferred embodiment the liquid cosmetic composition comprising chitosan modulates a function of the microbiota.

In another preferred embodiment the liquid cosmetic composition differentially promotes the growth of one or more microbial taxa relative to another microbial taxa. In a more preferred embodiment, the liquid cosmetic composition promotes the growth of one or more beneficial microbial taxa relative to one or more pathogenic microbial taxa. In another more preferred embodiment, the liquid cosmetic composition promotes the growth of one or more beneficial microbial taxa while at the same time does not harm the one or more pathogenic microbial taxa. In another more preferred embodiment, the liquid cosmetic composition promotes the growth of one or more beneficial microbial taxa while at the same time the growth of the one or more pathogenic microbial taxa is inhibited. In still another preferred embodiment, the liquid cosmetic composition comprising chitosan does not harm the growth of one or more beneficial microbial taxa but inhibits growth of one or more pathogenic microbial taxa.

The beneficial microbial taxa comprises Staphylococcus epidermidis, Staphylococcus mitis, Staphylococcus capitis, Corynebacterium specs., Propionibacterium acnes, Malassezia pachydermatis, Streptococcus spec., Streptococcus spec., Lactobacillus spec., Micrococcus spec. and Bacillus spec.,

The pathogenic microbial taxa comprises Staphylococcus aureus, Staphylococcus epidermidis, Pseudomonas aeruginosa, Enterococcus faecalis/faecium, E. coli, Klebsiella pneumoniae, Candida albicans, Microsporum canis, Acinetobacter baumanii, Staphylococcus intermedius and Staphylococcus pseudointermedius.

In some cases, beneficial taxa can transform to pathogenic taxa depending on their quantity, i.e. if occurring in increased numbers as compared to a healthy state of the microbiome. In this embodiment, pathogenic taxa as described above can additionally comprise Propionibacterium, Streptococcus spec. (in case of acne for example) or Propionibacterium, Corynebacterium, Staphylococcus spec., Streptococcus spec., in particular Staphylococcus aureus and Staphylococcus epidermidis (in case of psoriasis for example).

The Liquid Composition Comprising Chitosan

In a second aspect, the present invention relates to a liquid composition comprising chitosan which may be a cosmetic composition or pharmaceutical composition depending on the use. Said this, this liquid composition can be used for the cosmetic purposes described above and herewithafter, including pharmaceutical applications, such as, e.g., treatment of dysbiosis.

In one embodiment, the liquid composition comprises one further agent.

In another embodiment, the at least one further agent is selected from a COSMOS certified cosmetical component selected from the list as presented under the link http://www.cosmos-standard-rm.org/verifmp.php. This list is herewith incorporated by reference.

In a preferred embodiment, the at least one further agent is selected from the group comprising urea, glycolic acid, glyoxylic acid, glycerol, pentylene glycol, lactic acid, ascorbic acid, pyroglutamic acid, citric acid, tartaric acid, fumaric acid, succinic acid, malic acid, mandelic acid, aloe vera, rosa gallica, hyaluronic acid, salicylic acid, gallic acid, cellulose and derivatives thereof, pectin and derivatives thereof, gummi arabicum, dextrines, cyclodextrines, xanthan gum, thiocyanate, amino acids, sorbic acid, sodium chloride or combinations thereof.

In a preferred embodiment, the dextrine is a corn, tapioca, rice, potato, wheat or sorghum dextrine.

In a preferred embodiment, the cellulose or derivative thereof is a hydroxyethylcellulose including Natrosol™ or hydroxymethylcellulose.

In a preferred embodiment, the cyclodextrine is an acetyl, dimaltosyl, hydroxyethyl, maltosyl or methyl cyclodextrine.

In another preferred embodiment the cyclodextrine is an alpha, beta or gamma cyclodextrine.

In a preferred embodiment, the at least one further agent is an alpha hydroxyl carboxylic acid having 2 to 6 carbon atoms.

In another preferred embodiment the liquid composition is produced under protection gas, to prevent oxidation, and the product is packed in a primary package which does block oxygen from entering the primary package.

In another preferred embodiment the liquid composition is produced under protection gas, to prevent oxidation, and the product is packed in a primary package which does block oxygen from entering the primary package and will block air to enter the package to replace the dispensed volume (airless dispensing principle) In another preferred embodiment the liquid composition is packed in a primary package with airless dispensing principle.

In yet another preferred embodiment, the at least one further agent is selected from the group consisting of pentylene glycol, glycolic acid, glycerol, urea, thiocyanate or lactic acid or combinations thereof.

In yet another preferred embodiment, the at least one further agent is selected from the group consisting of glycolic acid, pentylene glycol, glycerol, urea, lactic acid or combinations thereof.

In another preferred embodiment, the at least one further agent is selected from the group consisting of glycolic acid, glycerol and lactic acid or combinations thereof.

In another preferred embodiment, the at least one further agent is selected from the group consisting of glycolic acid, pentylene glycol and lactic acid or combinations thereof

In another preferred embodiment, the at least one further agent is selected from the group consisting of glycolic acid, urea and lactic acid or combinations thereof.

In a particularly preferred embodiment, the at least one further agent comprises glycolic acid.

In a particularly preferred embodiment, the at least one further agent comprises lactic acid.

In a particularly preferred embodiment, the at least one further agent comprises glycolic acid and lactic acid.

In another particularly preferred embodiment, the at least one further agent comprises glycolic acid, lactic acid, pentylene glycol, urea and glycerol.

In another embodiment, the liquid composition comprises drugs.

In another embodiment, the liquid composition comprises particles.

In another embodiment, the liquid composition comprises fibres.

In another embodiment, the liquid composition comprises vesicles.

In another preferred embodiment, the molar ratio between chitosan monomers and acid groups of the acids or alpha hydroxyl carboxylic acids having 2 to 6 carbon atoms is between 1:1 and 1:1.1.

In another embodiment, the composition comprises a water/glycerol mixture, preferably a water/glycerol mixture with more than 20% (w/w) glycerol as solvent.

In another embodiment, the composition comprises a water/ethylenglycol mixture, preferably a water/ethylenglycol mixture with more than 20% (w/w) ethylenglycol as solvent.

In another embodiment, the composition comprises a water/ethylenglycol mixture, preferably a water/ethylenglycol mixture with more than 20% (w/w) ethylenglycol as solvent.

In another embodiment, the composition comprises a water/polyethylenglycol mixture, preferably a water/polyethylenglycol mixture with more than 10% (w/w) polyethylenglycol as solvent.

In another embodiment, the composition comprises a water/polypropylenglycol mixture, preferably a water/polypropylenglycol mixture with more than 10% (w/w) polypropylenglycol as solvent.

In another embodiment, the composition comprises a water/ethanol mixture, preferably a water/ethanol mixture with more than 20% (w/w) ethanol as solvent.

In another embodiment, the composition comprises a water/propanol mixture, preferably a water/propanol mixture with more than 20% (w/w) propanol as solvent.

In another embodiment, the composition comprises a water/isopropanol mixture, preferably a water/isopropanol mixture with more than 20% (w/w) isopropanol as solvent.

In another preferred embodiment, the liquid composition further comprises an emulsifier, surfactant or wetting agent. The emulsifier, surfactant or wetting agent helps to provide a homogenous distribution of the composition on the skin so that a thin and homogenous (cosmetic) membrane can form. Emulsifiers, surfactants and wetting agents are commonly known in the art. In a preferred embodiment, the emulsifier, surfactant or wetting agent is selected from the group consisting of polysorbates, alkyl amido betaines and alcohol polyglucosides or combinations thereof. In a particularly preferred embodiment, the wetting agent is a polysorbate, more preferably is Polysorbate 20.

In a preferred embodiment, the liquid composition further comprises a humectant. The humectant keeps the skin moist and thus improves the condition of dry skin or irritated skin.

Examples of suitable humectants nonexclusively include: 1) water soluble liquid polyols selected from the group comprising glycerol, propylene glycol, hexylene glycol, butylene glycol, pentylene glycol, dipropylene glycol and mixtures thereof; 2) hyaluronic acid; 3) polyalkylene glycol of the formula I.: HO—(R″O)_{b}—H, wherein R″ is an alkylene group having from about 2 to about 4 carbon atoms and b is an integer of from about 1 to about 10; 4) polyethylene glycol ether of methyl glucose of formula II.: CH₃—CH₆H₁₀O₅—(OCH₂CH₂)_{c}—OH, wherein c is an integer from about 5 to about 25; 5) urea; 6) fructose; 7) glucose; 8) honey; 9) lactic acid; 10) maltose; 11) sodium glucuronate; 12) pyroglutamic acid and its salts; 13) amino acids; 14) dexpanthenol; and 15) mixtures thereof, with pentylene glycol or glycerol being the preferred humectant.

In another preferred embodiment, the chitosan's degree of acetylation is 15% or less.

In another preferred embodiment, the chitosan's degree of acetylation is 10% or less.

In yet another preferred embodiment, the chitosan's degree of acetylation is even more preferably 5% of less.

In a particularly preferred embodiment, the chitosan's degree of acetylation is 2.5% or less.

In a particularly preferred embodiment, the chitosan's degree of acetylation is 2.0% or less.

The lower degree of acetylation is particularly suitable to mechanically protect the skin from harmful external stresses, such as undesirable microorganisms to settle on the skin, and at the same time provide a suitable environment for taking care of the skin. Also, lysozymal biodegradation of the chitosan or its dissolution can be limited or prevented.

The degree of acetylation can be obtained by means of ¹H NMR spectroscopy as, e.g., disclosed in Lavertu et al., “A validated ¹H NMR method for the determination of the degree of deacetylation of chitosan”, J. Pharm. Biomed. Anal. 2003, 32, 1149. “Deacetylated native chitosan” in the context of the present invention refers to chitosan that is both native and deacetylated according to the above definitions.

The preferred chitosan can be prepared by a method that involves at least two deacetylation steps. Two deacetylation steps are separated (and thus distinguished from a single deacetylation step) at least by a washing step in which by-products of the deacetylation, such as acetate, are at least partly removed. Preferably, at least one, more preferably all deacetylation steps are hydrolysis steps. A hydrolysis step may involve mixing the chitosan with a solution of a hydroxide such as sodium hydroxide. Preferably, during a hydrolysis step, the chitosan is exposed to a temperature higher than room temperature, e.g. 100° C. Preferably, at the end of each deacetylation step, the chitosan is washed, e.g. in water. Moreover, at least at the end of the last deacetylation step, preferably at the end of each deacetylation step, the chitosan is dried.

In certain embodiments of the invention, between two deacetylation steps an acetylation step is performed. A preferred acetylation step may involve mixing the chitosan or acidic chitosan solution with an organic solvent, followed by treatment with a carboxylic anhydride at room temperature. Preferably, at the end of the acetylation step, the acetylated chitosan is washed and dried.

In a preferred embodiment, the chitosan is preparable by a method that involves at least two deacetylation steps.

It is a further preferred embodiment that the liquid composition is an aqueous solution, i.e. it comprises water as the mixture medium or solvent, respectively. The chitosan is dissolved in the aqueous solution and may only form a (cosmetic) membrane after application of the cosmetic solution onto the subject's skin. In another preferred embodiment, the liquid cosmetic composition is topically applied to the skin in form of an emulsion, dispersion, suspension, serum, gel, a solution, a sprayable liquid, aerosol or foam.

In a particularly preferred embodiment, the liquid cosmetic composition is topically applied to the skin in form of a gel. The gel is applied in the form of a cosmetic film which then transforms into a (cosmetic) membrane due to the precipitation of the chitosan and possibly due to evaporation of the liquid of the gel.

In another preferred embodiment, the chitosan is native chitosan.

In another preferred embodiment, the chitosan or salt thereof is not a chitosan derivative, such as chitosan arginine amide.

Preferred salts of chitosan are those derived from the dissolution of a chitosan such as native chitosan, in an inorganic acid, such as hydrochloric acid, or an organic acid selected from the group of monobasic or multibasic organic acids having 2 to 12 carbon atoms and a first pKa value between 1 and 5. Particularly preferred salts are the salts of citric acid, lactic acid, glycolic acid, glyoxylic acid, malic acid, succinic acid, fumaric acid, pyroglutamic acid, mandelic acid, oxalic acid, tartaric acid, salicylic acid and ascorbic acid. Particularly preferred salts are the salts of lactic acid and glycolic acid or a combination thereof.

It is preferred that the chitosan according to the invention is a polymer of formula (I)

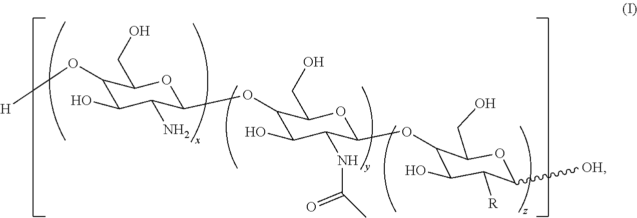

wherein x and z are independently an integer from 5 to 25000,

y represents an integer from 1 to 25000, and

R represents an —NH₂ group or —NH₃⁺CH₃CH(OH)C(O)O⁻ or —NH₃⁺ HOCH₂C(O)O⁻,

wherein the sequence of the units contained in the square brackets and having the indices x, y, and z can be freely chosen. It is preferred that x and z, independently of each other, represent integers from 5 to 20,000, preferably from 6 to 15,200, more preferably from 7 to 13,000. It is also preferred that y is an integer from 1 to 25,000, preferably from 1 to 20,000 and even more preferably from 1 to 15,000.

In another preferred embodiment, the liquid composition of the invention has a viscosity of from about 1 mPas to about 1000 mPas.

In yet another preferred embodiment, the liquid composition of the invention has a viscosity from about 10 mPas to about 1000 mPas preferably from about 10 mPas to about 800 mPas. The viscosity of the composition may for example be determined using a glass capillary viscometer.

In another embodiment, the liquid composition does not contain ethanol and/or isopropanol.

In yet another embodiment, the concentration of the chitosan of the liquid composition is at least 0.01% (w/w) based on the total weight of the liquid composition. In another preferred embodiment, the concentration of the chitosan of the liquid composition is at least 0.1% (w/w) based on the total weight of the liquid composition. In another preferred embodiment, the concentration of the chitosan of the liquid composition is at least 2.0% (w/w) based on the total weight of the liquid composition.

In a further embodiment, the concentration of the chitosan of the liquid composition is no more than 15% (w/w) based on the total weight of the liquid composition.

In a further embodiment, the concentration of the chitosan of the liquid composition is no more than 10% (w/w) based on the total weight of the liquid composition.

In a further embodiment, the concentration of the chitosan of the liquid composition is no more than 5% (w/w) based on the total weight of the liquid composition.

In a further embodiment, the concentration of the chitosan of the liquid composition is no more than 4% (w/w) based on the total weight of the liquid composition.

In a further embodiment, the concentration of the chitosan of the liquid composition is no more than 3% (w/w) based on the total weight of the liquid composition.

In another preferred embodiment, the concentration of the chitosan of the liquid composition is from 0.01% to 15% (w/w) based on the total weight of the liquid composition.

In a preferred embodiment, the concentration of the chitosan of the liquid composition is from 0.01% to 10% (w/w) based on the total weight of the liquid composition.

In another preferred embodiment, the concentration of the chitosan of the liquid composition is from 0.01% to 5% (w/w) based on the total weight of the liquid composition.

In a further preferred embodiment, the concentration of the chitosan of the liquid composition is from 0.1% to 4% (w/w) based on the total weight of the liquid composition.

In another preferred embodiment, the concentration of the chitosan of the liquid composition is from 2% to 4% (w/w) based on the total weight of the liquid composition.

In another preferred embodiment, the concentration of the chitosan of the liquid composition is from 1% to 3% (w/w) based on the total weight of the liquid composition.

In a preferred embodiment, the concentration of glycolic acid of the liquid composition is from 0.01% to 3% (w/w) based on the total weight of the liquid composition.

In a more preferred embodiment, the concentration of glycolic acid of the liquid composition is from 0.1% to 1% (w/w) based on the total weight of the liquid composition.

In a preferred embodiment, the concentration of lactic acid of the liquid composition is from 0.01% to 3% (w/w) based on the total weight of the liquid composition.

In a more preferred embodiment, the concentration of lactic acid of the liquid composition is from 0.1% to 2% (w/w) based on the total weight of the liquid composition.

In an even more preferred embodiment, the liquid composition comprises

0.05% to 15% (w/w) chitosan,

0.0% to 9% (w/w) glycolic acid and

0.0% to 9% (w/w) lactic acid,

based on the total weight of the liquid composition.

In an even more preferred embodiment, the liquid composition comprises

0.1% to 15% (w/w) chitosan,

0.1% to 9% (w/w) glycolic acid and

0.1% to 9% (w/w) lactic acid,

based on the total weight of the liquid composition.

In an even more preferred embodiment, the liquid composition comprises

0.1% to 15% (w/w) chitosan,

0.1% to 3% (w/w) glycolic acid and

0.1% to 3% (w/w) lactic acid,

based on the total weight of the liquid composition.

In an even more preferred embodiment, the liquid composition comprises

0.20 to 4.0% (w/w) chitosan,

0.0 to 2.5% (w/w) glycolic acid and

0.0 to 2.5% (w/w) lactic acid,

based on the total weight of the liquid composition.

In an even more preferred embodiment, the liquid composition comprises

0.01% to 4% (w/w) chitosan,

0.1% to 2.0% (w/w) glycolic acid and

0.1% to 2.2% (w/w) lactic acid,

based on the total weight of the liquid composition.

In another embodiment, the composition comprises

0.01 to 4.0% (w/w) chitosan,

0.005 to 2.5% (w/w) glycolic acid,

0.005 to 2.5% (w/w) lactic acid,

based on the total weight of the liquid cosmetic composition.

In an even more preferred embodiment, the liquid composition comprises

2.0 to 4.0% (w/w) chitosan,

0.3 to 2.2% (w/w) glycolic acid and

0.3 to 2.2% (w/w) lactic acid,

based on the total weight of the liquid composition.

In another embodiment, the composition comprises

2.7 to 3.3% (w/w) chitosan,

0.7 to 1.3% (w/w) glycolic acid,

0.7 to 1.3% (w/w) lactic acid,

based on the total weight of the liquid cosmetic composition.

In an even more preferred embodiment, the liquid composition comprises

2.0 to 4.0% (w/w) chitosan,

0.3 to 2.2% (w/w) glycolic acid and

0.3 to 2.2% (w/w) lactic acid,

based on the total weight of the liquid composition, wherein the degree of acetylation of the chitosan is 15%.

In an even more preferred embodiment, the liquid composition comprises

2.0 to 4.0% (w/w) chitosan,

0.3 to 2.2% (w/w) glycolic acid and

0.3 to 2.2% (w/w) lactic acid,

based on the total weight of the liquid composition, wherein the degree of acetylation of the chitosan is 2%.

In an even more preferred embodiment, the liquid composition comprises

0.20 to 4.0% (w/w) chitosan,

0.0 to 2.5% (w/w) glycolic acid and

0.0 to 2.5% (w/w) lactic acid,

based on the total weight of the liquid composition, wherein the composition comprises no preservative.

0.20 to 4.0% (w/w) chitosan,

0.3 to 2.2% (w/w) glycolic acid and

0.3 to 2.2% (w/w) lactic acid,

based on the total weight of the liquid composition, wherein the composition comprises no preservative.

In another preferred embodiment, the liquid composition further comprises a preservative. It is particularly preferred that said preservative is sorbic acid in its free acid form or a cosmetically acceptable salt thereof. The sorbic acid or cosmetically acceptable salt thereof is then used in a concentration commonly used for preservatives. It is even more particularly preferred that the liquid composition comprises sorbic acid in a concentration ranging from 0.02% to 0.2% (w/w) based on the total weight of the liquid composition. In another preferred embodiment, the preservative is potassium sorbate. In another preferred embodiment, the liquid composition comprises no preservative.

Colorants or pigments can be added to the liquid cosmetic composition to a achieve a desired color for application to the skin. Such colorants or pigments are known and the concentrations required to achieve a desired coloring are readily determinable. Pigments maybe inorganic or organic. Inorganic pigments include iron oxides (red, black, brown colors), manganese violet, ultramarines (green, blue, pink, red, or violet aluminum sulfosilicates), aquamarines, copper powder, mica, clays, silica and titanium dioxide. Organic pigments are generally various types including azo, indigoid, triphenylmethane, anthraquinone and xanthine dyes which are designated as D&C and FD&C blues, browns, greens, oranges, reds, yellows etc. Each of these pigments may further have several different trade names or be present in mixed compositions.

In certain embodiments, the liquid composition can contain a colorant or a pigment in a concentration of 0% to 30% (w/w), 1% to 20% (w/w), 2% to 15% (w/w) or 5% to 15% (w/w).

Fragrances or scavengers can be added to the liquid cosmetic composition to achieve a desired smell for the application on the skin. Such fragrances are known and the concentrations required to achieve a desired smell are readily determinable.

In certain embodiments, the liquid composition can contain a fragrance in a concentration of 0% to 5% (w/w), 0.001% to 1% (w/w), 0.001% to 0.5% (w/w) or 0.001% to 0.1% (w/w).

In certain embodiments, the liquid composition can contain an odor neutralizer in a concentration of 0% to 5% (w/w), 0.01% to 3% (w/w), 0.01% to 1% (w/w) or 0.01% to 0.5% (w/w).

In certain embodiments, the liquid composition can contain an odor absorber in a concentration of 0% to 5% (w/w), 0.01% to 3% (w/w), 0.01% to 1% (w/w) or 0.01% to 0.5% (w/w).

In certain embodiments, the liquid composition can contain sunblockers (organic chemical compounds, inorganic particulates, organic particulates with UV filtering capabilities).

The Membrane Formed by the Liquid Composition Comprising Chitosan

It often takes a long time for agents to be taken up by the skin or it is desirable that a cosmetic or medical agent remains at the immediate surface of the skin. If these agents are applied as a cream or ointment and remain on the skin for a long time, the skin has a fatty shine and feels oily, fatty or sticky. On the other hand, if the same agents are applied as a gel or a serum, the agent may immediately be soaked into deeper layers of the skin and cannot protect or provide care of the upper skin layers.

It was surprisingly found that the liquid composition of the present invention forms a membrane which provides advantages of cream and ointment as well as of gel and serum: the membrane on skin provides no or substantially no fatty shine and no oily or sticky feeling of the skin but also lets a further agent remain at the skin surface.

In one embodiment, the liquid composition is applied topically to form a membrane. In one embodiment, the formed membrane has a thickness of 0.001 nm to 50 μm, preferably 0.001 nm to 10 μm and more preferably from 0.001 nm to 1 μm. In a preferred embodiment, the formed membrane has a thickness of 0.001 nm to 50 μm, preferably 0.001 nm to 10 μm and more preferably from 0.001 nm to 1 μm. In another preferred embodiment, the formed membrane has a thickness of 1 nm to 50 μm, preferably 1 nm to 10 μm and more preferably from 1 nm to 1 μm. In another preferred embodiment, the formed membrane has a thickness of 10 nm to 10 μm, preferably 10 nm to 1 μm and more preferably from 10 nm to 100 nm or 4 nm to 50 nm.

The membrane can form a stable support for conventional make-up which is applied following the membrane's formation with no disturbing optical impact. The membrane also allows for bidirectional transport of e.g. water, nutrients, oxygen, carbondioxide and other gases and may e.g. act as a slow release storage for other active molecules such as drugs, pharmaceutical compositions, or vitamins for example.

As the liquid composition comprising chitosan according to the invention forms a membrane, when applied topically, the same embodiments as described for the liquid composition also apply to the membrane. Thus, the membrane has the same properties of influencing the microbiome on a subject's ectodermal tissue as stated above for the liquid composition comprising chitosan.

It was further surprisingly found that the membrane resulting from the application of the inventive liquid composition can serve as a stable support for conventional make-up with no disturbing optical impact. Following formation of the membrane on the skin area treated, such area can become subject of conventional make-up application. Membrane covered skin areas and untreated skin do not result in optical heterogeneity or optical effects along the boundary between covered skin and uncovered skin.

The chitosan or salt thereof is present in a precipitated form and preferably the main component of the membrane. Further, the chitosan or salt thereof functions as carrier component of the membrane.

It was surprisingly found that if the liquid composition is applied onto the skin as described above, the resulting membrane provides a pH of 3.5 to 6.5, which is particularly advantageous since the pH of the membrane is similar to the pH of the skin. This means that the membrane covering the skin provides very good conditions for skin care applications so that the skin is no further stressed by the application of the at least one further agent, e.g. when applied with a membrane having pH 7.

In a preferred embodiment, the pH of the membrane is from about 4.0 to about 6.0. In another preferred embodiment, the pH of the membrane is from 4.0 to about 5.0. In another preferred embodiment, the pH of the membrane is from about 4.5 to about 5.5. In another preferred embodiment, the pH of the membrane is from about 5.0 to about 6.0. It was surprisingly found that such a membrane can be formed as it was previously assumed that chitosan only precipitates when the pH of the dissolved chitosan in a liquid composition is increased above 6.3. In another embodiment, the membrane has a pH of from about 4.0 to about 6.5, preferably of 4.0 to 6.0, even more preferably of from about 4.0 to about 5.4 and most preferably of from about 4.0 to about 5.2 or even of from about 4.0 to about 5.0.

One way to achieve the precipitation of chitosan would thus be by adding an alkaline solution to the composition or by using a volatile acid, such as acetic acid, as the solvent for chitosan which would evaporate after application of the respective dissolution of chitosan onto the skin, thereby increasing the pH above 6.3 to let the chitosan precipitate. It was surprisingly found that the addition of an alkaline solution or a dissolving the chitosan in a volatile acid in order to increase the pH above 6.3 is not necessary when the membrane is obtained by applying the liquid composition of the present invention to the skin of a subject.

Therefore, in a preferred embodiment, the liquid composition of the invention does not comprise a volatile acid such as acetic acid. The use of volatile acids such as acetic acid is not desirable in a composition as the odor of the volatile acid may be considered unpleasant by the consumer.

In a preferred embodiment, the liquid composition has a pH of about 4.5 to about 6.0. In another preferred embodiment, the liquid composition has a pH of about 4.5 to about 5.5. The low pH of the liquid composition is necessary to dissolve the chitosan or salt thereof for the application to the skin.

It is thus another preferred embodiment that the membrane is homogenous.

In another preferred embodiment, the membrane covers at least an area of 1 mm×1 mm and preferably at least 2 mm×2 mm. Thus, the membrane covers a larger area of skin than e.g. a single nanoparticle where the chitosan encapulates an agent which when applied onto the skin does not result into a homogenous layer of a membrane. Instead, only small areas of “nanopatches” of nanocapsules on top of the skin. This use of chitosan nanoparticles has the disadvantage that an area of skin cannot be homogenously covered as with a membrane.

It is another preferred embodiment that the membrane forms within 5 minutes after the composition has been applied to the skin under standard conditions of 25° C. and 101.3 kPa.

In another preferred embodiment 0.1 μl-10 μl of the liquid composition are sufficient to be applied per 1 cm² of skin. In another preferred embodiment the liquid composition has dried on the skin within 10 s-300s, so that it will not become soaked by or transferred to materials which come into contact with the membrane on the skin.

It was surprisingly found that pH lowering with dilution and pH raising with concentration supports and accelerates the formation of the insoluble membrane on the skin surface with evaporation or uptake of solvent from the gel-like chitosan solution applied.

In another embodiment, the membrane comprises at least 10% (w/w) chitosan or a salt thereof based on the total weight of the membrane.

In another preferred embodiment, the membrane is permeable for the at least one agent. This way, the membrane functions as carrier for the at least one further agent but at the same time protects the skin under the membrane from undesired stress factors, such as an alkaline agent, UV irradiation, other chemicals or direct contact to clothes which may further irritate the skin.

In another preferred embodiment, the membrane formed by the liquid composition according to the invention is permeable for the at least one further agent. This way, the membrane functions as carrier for the at least one further agent. At the same time, the membrane formed by the liquid composition according to the invention can be covered with cosmetic suspensions, emulsions, foams, liquids, gels and oils not comprising the liquid composition according to the invention.

In another preferred embodiment, the skin will be treated first with cosmetic suspensions, emulsions, foams, liquids, gels and oils not comprising the liquid composition according to the invention, before the liquid composition according to the invention is applied which forms a membrane.

In another preferred embodiment, the skin will be treated first with cosmetic suspensions, emulsions, foams, liquids, gels and oils not comprising the liquid composition according to the invention, before the liquid composition according to the invention is applied which forms a membrane. The resulting membrane can then at the same time be covered with cosmetic suspensions, emulsions, foams, liquids, gels and oils.

In another preferred embodiment, the membrane formed by the liquid composition according to the invention is permeable for the at least one further agent. This way, the membrane functions as carrier for the at least one further agent but at the same time can be covered with medical suspensions, emulsions, foams, liquids, gels and oils comprising drugs.

In another preferred embodiment, the skin will be treated first with medical suspensions, emulsions, foams, liquids, gels and oils comprising drugs, before the liquid composition according to the invention is applied which forms a membrane.

In another preferred embodiment, the skin will be treated first with medical suspensions, emulsions, foams, liquids, gels and oils comprising drugs, before the liquid composition according to the invention is applied which forms a membrane. The resulting membrane can then at the same time be covered with medical suspensions, emulsions, foams, liquids, gels and oils comprising drugs.

The Use of the Liquid Cosmetic Composition for Skin Care Applications

In a fourth aspect, the present invention relates to the use of a liquid composition as described in the aforementioned embodiments for skin care applications, i.e. for a cosmetical purpose, wherein the liquid composition is applied topically on an area of a subject's skin. This results in the formation of a membrane according to the invention which is of cosmetical purpose and will be termed cosmetic membrane in the following.

It is preferred that the skin care is a care of abrasions, care of cuts, care of pimples, care of blisters, care of stings, care of burns, anti-ageing application, care of irritated skin, care of radiated skin, care of laser treatments, care of plasma treatments, care of tattoos and removal of tattoos, care of skin suffering from viral infection, care or prevention of skin peeling, care or prevention of radiation induced skin changes, care or prevention of formation of skin scar tissue, prevention of dry skin, prevention of fatty skin, prevention of cracking of skin, prevention of stretch marks, reduction of wrinkling of the skin, protection of thin skin or a skin cleansing application or prevention of accompanying symptoms thereof.

It is particularly preferred that the skin care application is anti-ageing application, care of irritated skin, care of dry skin, reduction of wrinkling of the skin, protection of thin skin, prevention of cracking of skin, care of stretch marks or a skin cleansing application.

It is particularly preferred that the skin care application is care of abrasions, care of cracks, care of cuts, care of pimples, care of blisters, care of stings, care or prevention of formation of skin scar tissue or prevention of accompanying symptoms thereof.

Accompanying symptoms of any of the above skin care applications may be surface pain, itching, wet feeling, smell, roughness, crackiness, fissures, dryness, ugliness, peeling or other unpleasant stress symptoms or combinations thereof.

In a preferred embodiment, the skin care application is the care of irritated skin or dry skin. In another preferred embodiment, the liquid cosmetic composition is used for the care of fatty skin.

In a third aspect, the present invention further relates to the use of a cosmetic membrane for skin care applications. It is preferred that the skin care is a care of abrasions, care of cuts, care of pimples, care of blisters, care of stings, care of burns, anti-ageing application, care of irritated skin, care of radiated skin, care of laser treatments, care of plasma treatments, care of tattoos and removal of tattoos, care of skin suffering from viral infection, care or prevention of skin peeling, care or prevention of radiation induced skin changes, care or prevention of formation of skin scar tissue, prevention of dry skin, prevention of fatty skin, prevention of cracking of skin, prevention of stretch marks, reduction of wrinkling of the skin, protection of thin skin or a skin cleansing application or prevention of accompanying symptoms thereof.

It is particularly preferred that the skin care application is anti-ageing application, care of irritated skin, care of dry skin, reduction of wrinkling of the skin, protection of thin skin, prevention of cracking of skin, care of stretch marks, or a skin cleansing application.

It is particularly preferred that the skin care application is care of abrasions, care of cracks, care of cuts, care of pimples, care of blisters, care of stings, care or prevention of formation of skin scar tissue or prevention of accompanying symptoms thereof.

Accompanying symptoms of any of the above skin care applications may be surface pain, itching, wet feeling, smell, roughness, crackiness, fissures, dryness, ugliness, or other unpleasant stress symptoms or combinations thereof.

In a preferred embodiment, the skin care application is the care of irritated skin or dry skin.

In another preferred embodiment, the cosmetic membrane is used for the care of fatty skin.

In another aspect, the present invention relates to the use of the liquid composition as a medical product.

The Disinfectant

In preferred embodiment of the invention, the liquid composition comprising chitosan comprises a disinfectant. The disinfectant comprises alcohol, aldehyde, iodine, chlorine, quarternary ammonia compound, peroxide, amphotenside, phenol, alkylamine, acid and/or base. The simultaneous provision of an disinfectant is particularly advantageous if disinfection is necessary. The liquid composition comprising chitosan promotes re-establishment of a healthy microbiome after disinfection and/or inhibits growth of pathogenic microbes.

Alcohol disinfectants comprise ethanol, propanol and isopropanol. Aldehyde disinfectants comprise formaldehyde, glutaraldehyde and glyoxal. Iodine compounds include compounds which release iodine and are synonymous to iodophores. Chlorines include free chlorine or compounds that split off chlorine such as hypochlorite releasing compounds (e.g. alkali hypochlorite, hypochlorous acid). Quarternary ammonia compounds include guanides, biguanides, guanidine salts and bisbiguanides such as chlorhexidine, polyhexamethyl biguanide, polyhexamethylene guanidine hydrochloride, polyhexamethylene guanidine hydrophosphate, and poly[2-(2-ethoxy)-ethoxyethyl]-guanidinium chloride. Peroxides include hydrogen peroxide, peracetic acid, benzoyl peroxide, sodium perborate, potassium permanganate and perbenzoic acid. Alkyl amines include primary, secondary and tertiary alkyl amines. An exemplary alkylamine is N,N-bis-(3-aminopropyl) lauryl amine. Acids include organic and inorganic acids. Organic acids include formic acid, phenylacetic acid, acetic acid, citric acid and propionic acid. Further acids include protonated carboxylic acids (e.g. heptanoic, octanoic, nonanoic, decanoic, undecanoic acids), acid anionics (e.g. alkylaryl sulfonic acids, aryl sulfonic acid, alkyl sulfonic acids, alkylaryl sulfuric acid, aryl sulfuric acid, alkyl sulfuric acid, alkylaryl sulfuric acid) and chlorine dioxide from alkali chlorite by an acid activator. Bases include sodium hydroxide, potassium hydroxide and calcium hydroxide. Phenolic disinfectants may be chosen from 2,4,4″-trichloro-2′-hydroxydiphenylether, which is known commercially as triclosan and 4-chloro-3,5-dimethyl phenol, which is also known as PCMX. Traditional disinfectants further include copper sulfate, zinc sulfate and sulfamethazine. Amphotensides include N-Alkyl-di(-aminoethyl)-glycine and N-Alkylaminopropylglycine.

The disinfectant can further be one of the following trade names: ACTIL Händedesinfektion, A H D 2000, Alcoman, ALCOSYN, Amosept, APESIN, APESIN, APESIN, Aseptom, Aseptom, Aseptom, Aseptom, Aseptom, Aseptoman plus, Aseptoman viral, Aseptopur, Aseptopur Viral, Bojasept, C 20, C 25, calgonit Des-H, calgonit Händedesinfektion, Chirosyn Händedesinfektion, CimoCid, CimoCid Sensitive, CimoSept Hände, CimoSkin, Decontaman, Dermasept, Dermocol Gel New, Dermocol New Colorless, Descoderm, Descoderm viral, Desderman pure, Desderman care, Desmanol N, Desmanol pure, Desmanol care, DESTAsept DERM pro, DESTAsept MAN pro, DESTAsept rapid N, Ethasept, FAVORIT Händedesinfektion, FAVORIT Händedesinfektion+gel, Halasept 792, Halasept 820 Gel, Halasept 880 E, HD 410, HD 412 essential, Herwe Dermasept N Gel, Herwe Dermasept N Liquid, Hospisept, Kaniderm, Kaniderm Premium, Kaniderm Protect, kodan Tinktur forte, L+R handdisinfect blue, L+R handdisinfect gel, L+R handdisinfect green, Lerasept® HD, Manocid, Manorapid basic, Manorapid Plus, Manorapid r.f.u., Manorapid Synergy, ManuPep CARE, ManuPep GEL, ManuPep Händedesinfektion, Manusept basic, marina-KC Opydes, MEDIman H2, Mucasept Plus, MYXAL® SEPT 70, MYXAL® SEPT 80, MYXAL® SEPT Ge, Neosept, Neoseptin, octeniderm, Op Sept, Op Sept Basic, Pluraman GEL, Pluraman SOFT, Poly-Alcohol Hände, Antisepticum, Promanum pure, Protectasept Haut- and Händedesinfektion, PuraDES PentaMAN B, PuraDES Pentra MAN, PuraDES TeraMAN GEL, PuraDES TetraMAN, PuraDES TetraMAN B, REGOskin DS 4051, Sanocid, Sanocid Gel, Sanocid Plus, sensiva Händedesinfektion, septDES FOAM, septDES FOAMSOAP, septDES GEL, Septiderm, septLIQUID PLUS, septLIQU, ID SENSITIVE, Skinman clear, Skinman complete, Skinman complete pure, Skinman foam, Skinman soft, Skinman soft protect, Skinman soft protect FF, Skinsept F, SKINTASTIC Leocid Sept, Soft Care Des E, Soft Care Des E Foam, Soft Care Des E Spray, Soft Care Med, Softa-Man acute, Softa-Man pure, Softa-Man ViscoRub, Spitacid, Sterillium, Sterillium classic pure, Sterillium med, Sterillium Tissues, Sterillium Virugard, Sterillium® pure, Sure Instant Hand Sanitizer, triformin safeDIS, weigoman, weigoman parfümfrei, weigoman pure and Witty-Lavalin D.

The Ectodermal Tissue

In a preferred embodiment, the ectodermal tissue is the skin. In a more preferred embodiment, the ectodermal tissue is the epidermis. In another preferred embodiment, the ectodermal tissue is damaged epidermis, wherein the damage comprises sunburn, acne, cuts, abrasions, cuts, pimples, blisters, stings, burns, ageing, irritated skin, radiated skin, laser treated skin, plasma treated skin, a tattoo, the removal of a tattoo, skin peeling, scar tissue, dry skin, fatty skin, cracks, stretch marks or wrinkling. In a more preferred embodiment, the subject's ectodermal tissue, skin, epidermis or damaged epidermis has previously been sterilized or disinfected. Use of the liquid composition comprising chitosan is particularly advantageous after disinfection as the liquid composition comprising chitosan promotes re-establishment of a healthy microbiome after disinfection and/or inhibits growth of pathogenic microbes.

In another preferred embodiment, the ectodermal tissue is stressed epidermis, wherein the stress is caused by prostheses, endoprostheses, orthoses, exoskeletons, plasters, compression bandages, stockings, bandages, latex protection, massagers, masks for ventilation, apnea prevention, work- or protective clothing, gloves, pacifiers, tight clothes or shoes, disinfectants, cosmetic treatments, cosmetic products, preservatives, cleaning agents, sweat, lack of body hygiene, long lying or sitting, wound dressings, sunburns, burns, stings, radiation, laser treatment, plasma treatment, tattooing and/or removal of tattoos.

Preferably, the stress is caused by massagers, work- or protective clothing, gloves, pacifiers, tight clothes or shoes, cosmetic treatments, cosmetic products, preservatives, cleaning agents, sweat, lack of body hygiene, sunburns, burns, stings, radiation, laser treatment, plasma treatment, tattooing and/or removal of tattoos.

Work or protective clothing comprises high boots, airtight protective suits, masks, helmets, gloves and breathing masks. Latex protection comprises for example latex gloves.

Preferably, the stress is caused by prostheses, endoprostheses, orthoses, exoskeletons, plasters, compression bandages, stockings, bandages, latex protection, massagers, masks for ventilation, work- or protective clothing, gloves, long lying or sitting or wound dressings. The underlying reason for such stress limited moisture and gas exchange on the skin or epidermis.

Long lying or sitting may be due to hospitalization or sitting in a wheel chair. Symptoms include pressure sores and pressure ulcers. Thus, preferably, the stress may also be caused by pressure sores and pressure ulcers, more preferably by pressure sores.

Cosmetic skin treatments for example comprise peelings by abrasives, acids and laser treatment.

Stress caused by cleaning agents may be due to work, hobby, sports or house-keeping tasks in which the skin is in contact to cleaning agents.

In another aspect, the present invention provides for a kit comprising a liquid cosmetic composition according to any of the above embodiments and further comprising a disinfectant. In a preferred embodiment, the disinfectant is selected from the group comprising an alcohol, aldehyde, iodine, chlorine, quarternary ammonia compound, peroxide, amphotenside, phenol, alkylamine, acid and/or base.

The Composition Comprising Chitosan for the Treatment of Dysbiosis

The following embodiments relate to the fifth aspect of the present invention, i.e. a pharmaceutical composition comprising chitosan for the treatment of dysbiosis.

It was surprisingly found by the inventors that the liquid composition comprising chitosan as described above is also useful in the treatment of diseases or disorders which are associated with the microbiome such as in the treatment of dysbiosis. Thus, in the context of the present aspect, the liquid composition comprising chitosan is referred to as a pharmaceutical composition comprising chitosan in a liquid dosage form. The differential promotion of a healthy microbiome leads to the reduction or elimination of the potential for infections of other skin areas, infection of other individuals or contamination of other individuals, droplets and surfaces. Similar to the cosmetic application, the promotion of a healthy microbiome can be especially helpful after or simultaneous to disinfection or sterilisation, i.e. intentional elimination of the (healthy) microbiome which may be necessary prior to medical interventions such as surgery, injections or catheter applications.

In one embodiment, the pharmaceutical composition comprises chitosan in a liquid dosage form for use in treating a dysbiosis on a subject's ectodermal tissue. In another embodiment, the treatment of dysbiosis comprises modulating a microbial taxa on the ectodermal tissue of the subject. In a preferred embodiment the modulating a microbial taxa comprises an increase or decrease in the abundance of the taxa. In another preferred embodiment modulating a microbial taxa comprises an increase or decrease in the abundance of the taxa relative to the abundance of said microbial taxa in the absence of the pharmaceutical composition. In another preferred embodiment, modulating a microbial taxa comprises an increase or decrease of the abundance of the taxa relative to the abundance of a second microbial taxa.

In another preferred embodiment the pharmaceutical composition differentially promotes the growth of one or more microbial taxa relative to another microbial taxa. In a more preferred embodiment, the pharmaceutical composition promotes the growth of one or more beneficial microbial taxa relative to one or more pathogenic microbial taxa. In another more preferred embodiment, the pharmaceutical composition promotes the growth of one or more beneficial microbial taxa while at the same time does not harm the one or more pathogenic microbial taxa. In another more preferred embodiment, the pharmaceutical composition promotes the growth of one or more beneficial microbial taxa while at the same time the growth of the one or more pathogenic microbial taxa is inhibited. In still another preferred embodiment, the pharmaceutical composition comprising chitosan does not harm the growth of one or more beneficial microbial taxa but inhibits growth of one or more pathogenic microbial taxa.

The beneficial microbial taxa comprises Staphylococcus epidermidis, Staphylococcus mitis, Staphylococcus capitis, Corynebacterium specs., Propionibacterium acnes, Malassezia pachydermatis, Streptococcus spec., Streptococcus spec., Lactobacillus spec., Micrococcus spec. and Bacillus spec.,

The pathogenic microbial taxa comprises Staphylococcus aureus, Staphylococcus epidermidis, Pseudomonas aeruginosa, Enterococcus faecalis/faecium, E. coli, Klebsiella pneumoniae, Candida albicans, Microsporum canis, Acinetobacter baumanii, Staphylococcus intermedius and Staphylococcus pseudointermedius.

In some cases, beneficial taxa can transform to pathogenic taxa depending on their quantity, i.e. if occurring in increased numbers as compared to a healthy state of the microbiome. In this embodiment, pathogenic taxa as described above can additionally comprise Propionibacterium, Streptococcus spec. (in case of acne for example) or Propionibacterium, Corynebacterium, Staphylococcus spec., Streptococcus spec., in particular Staphylococcus aureus and Staphylococcus epidermidis (in case of psoriasis for example).

The pharmaceutical composition comprising chitosan can be any composition as described above under the item “The liquid composition comprising chitosan”. As the liquid composition is the same, all embodiments relating to the membrane which is formed by the liquid composition, as referred to under the item “The membrane formed by the liquid composition comprising chitosan” equally apply to the membrane formed by the pharmaceutical composition comprising chitosan.

In a particularly preferred embodiment, the pharmaceutical composition comprising chitosan for use in treating a dysbiosis further comprises glycolic acid and/or lactic acid. In a particularly preferred embodiment, the pharmaceutical composition comprising chitosan for use in treating a dysbiosis further comprises glycolic acid. In a particularly preferred embodiment, the pharmaceutical composition comprising chitosan for use in treating a dysbiosis further comprises lactic acid.

In a particularly preferred embodiment, the pharmaceutical composition comprising chitosan for use in treating a dysbiosis further comprises glycolic acid and lactic acid.

In an even more preferred embodiment, the pharmaceutical composition comprising chitosan for use in treating a dysbiosis comprises

0.010 to 4.0% (w/w) chitosan,

0.005 to 2.5% (w/w) glycolic acid,

0.005 to 2.5% (w/w) lactic acid,

based on the total weight of the pharmaceutical composition.

In an even more preferred embodiment, the pharmaceutical composition comprising chitosan for use in treating a dysbiosis comprises

0.20 to 4.0% (w/w) chitosan,

0.05 to 2.5% (w/w) glycolic acid,

0.05 to 2.5% (w/w) lactic acid,

based on the total weight of the pharmaceutical composition.

In still another more preferred embodiment, the pharmaceutical composition comprising chitosan for use in treating a dysbiosis comprises

0.20 to 4.0% (w/w) chitosan,

0.05 to 2.5% (w/w) glycolic acid,

0.05 to 2.5% (w/w) lactic acid,

based on the total weight of the pharmaceutical composition; wherein the degree of of acetylation of the chitosan is 2.5% or less and preferably 2.0% or less; and

wherein the pH of the liquid cosmetic composition is from about 4.0 to about 6.5.

In still another more preferred embodiment, the pharmaceutical composition comprising chitosan for use in treating a dysbiosis comprises

2.7 to 3.3% (w/w) chitosan,

0.5 to 1.3% (w/w) glycolic acid,

0.5 to 1.3% (w/w) lactic acid,

based on the total weight of the pharmaceutical composition; wherein the degree of of acetylation of the chitosan is 2.5% or less and preferably 2.0% or less; and wherein the pH of the liquid cosmetic composition is from about 4.0 to about 6.5.

In an even more preferred embodiment, the pharmaceutical composition comprising chitosan comprises a disinfectant. The disinfectant comprises alcohol, aldehyde, iodine, chlorine, quarternary ammonia compound, peroxide, amphotenside, phenol, alkylamine, acid and/or base. The simultaneous provision of an disinfectant is particularly advantageous if disinfection is necessary. The liquid composition comprising chitosan promotes re-establishment of a healthy microbiome, i.e. eubiosis, after disinfection and/or inhibits growth of pathogenic microbes. In general, the specific disinfectants as described above under the heading “The disinfectant” can be equally applied to the pharmaceutical composition comprising chitoson.

In one embodiment, the dysbiosis is idiopathic. Idiopathic means that the subject has no substantially observable cause of a dysbiosis. In another embodiment, the dysbiosis is associated with a disease, disorder or condition in a subject. In some embodiments, the disease, disorder or condition comprises an infectious disease, an inflammatory disease, a metabolic disease, an autoimmune disease or a cancer. Preferably, the disease, disorder or condition comprises an infectious disease, an inflammatory disease or an autoimmune disease. In some embodiments, the infectious disease is a viral, bacterial and/or fungal skin infection. Viral skin infections comprise herpes simplex (cold sores and genital herpes), herpes zoster (shingles), warts, and molluscum contagiosum.

Bacterial skin infections comprise cellulitis, erysipelas, impetigo, folliculitis, and furuncles and carbuncles. Cellulitis is an infection of the dermis and subcutaneous tissue that has poorly demarcated borders and is usually caused by Streptococcus or Staphylococcus species. Erysipelas is a superficial form of cellulitis with sharply demarcated borders and is caused almost exclusively by Streptococcus. Impetigo is also caused by Streptococcus or Staphylococcus and can lead to lifting of the stratum corneum resulting in the commonly seen bullous effect. Folliculitis is an inflammation of the hair follicles. Fungal skin infections comprise infections cause by yeasts (such as Candida or Malassezia furfur) or dermatophytes, such as Epidermophyton, Microsporum, and Trichophyton.

In some embodiments, the inflammatory disease comprises inflammatory bowel disease (IBD), ulcerative colitis (UC), Crohn's disease (CD), idiopathic inflammation of the small bowel, indeterminatal colitis, pouchitis, irritable bowel syndrome (IBS), necrotizing enterocolitis (NEC), intestinal inflammation, constipation, microscopic colitis, diarrhea, graft versus host disease (GVHD), allergies (e.g., food allergies), pseudomembranous colitis, indigestion, non-ulcer dyspepsia, diverticulosis, diverticulitis, ischemic colitis, radiation colitis, radiation enteritis, collagenous colitis, gastroenteritis, or polyps. In some embodiments, the metabolic disease comprises obesity, (insulin resistance) pre-diabetes, type II diabetes, high fasting blood sugar (hyperglycemia), metabolic syndrome, or a cardiovascular risk factor (e.g., high blood cholesterol, high LDL, high blood pressure (hypertension), high triglyceride levels, low HDL).

In some embodiments, the autoimmune disease comprises Crohn's disease, psoriasis, allergy, asthma, urticaria or atopic dermatitis. In some embodiments, the cancer is a cancer of the skin.

In general, diseases, disorders and conditions which are associated with dybiosis are described and can be found in “Skin Signs of Systemic Disease”, Saunders, 1998.

In some embodiments, the dysbiosis is associated with or a consequence of immunodeficiency or low immunity, immunosuppression, the administration of antibiotics, chemotherapeutic s, antibodies, cytokines, cell-therapeutics, therapeutic nucleic acids, immunesuppressants, burns or radiation of the skin, stress of the skin, the body or areas thereof.

In a preferred embodiment, the stress comprises stress due to prostheses, endoprostheses, orthoses, exoskeletons, plasters, compression bandages, stockings, bandages, latex protection, masks for ventilation, long lying or sitting or wound dressings. Long lying or sitting may be due to hospitalization or sitting in a wheel chair. Symptoms of long lying or sitting comprise pressure sores and pressure ulcers. Thus, preferably, the stress may also be caused by pressure sores and pressure ulcers. More preferably, the stress may be pressure ulcers.

In an even more preferred embodiment, the subject's ectodermal tissue, skin, epidermis, damaged or stressed epidermis, acute or chronic wound has previously been sterilized or disinfected. Put in other words, if the subject's skin has been disinfected with one of the above described disinfectants, the pharmaceutical composition comprising chitosan is immediately applied to the subject's skin after disinfection. If medical interventions are necessary after disinfection, then the pharmaceutical composition comprising chitosan is immediately applied to the subject's skin after the medical interventions which followed the initial disinfection. Optionally, another step of disinfection can occur after the medical interventions.

Further preferred embodiments of the present invention relate to:

1. A pharmaceutical composition comprising chitosan in a liquid dosage form for use in treating a dysbiosis on a subject's ectodermal tissue.

2. The pharmaceutical composition for use of 1, wherein treating the dysbiosis comprises modulating a microbial taxa on the ectodermal tissue of the subject.

3. The pharmaceutical composition for use of 2, wherein modulating a microbial taxa comprises an increase or decrease in the abundance of the taxa.

4. The pharmaceutical composition for use of 3, wherein modulating a microbial taxa comprises an increase of the abundance of a beneficial taxa relative to the abundance of a pathogenic taxa.

5. The pharmaceutical composition for use of 3, wherein modulating a microbial taxa comprises no substantial alteration of the abundance of a beneficial taxa relative to a decrease in the abundance of a pathogenic taxa.

6. The pharmaceutical composition for use of 4 or 5, wherein the beneficial microbial taxa comprises Staphylococcus epidermidis, Staphylococcus mitis, Staphylococcus capitis, Corynebacterium specs., Propionibacterium acnes, Malassezia pachydermatis, Streptococcus spec., Streptococcus spec., Lactobacillus spec., Micrococcus spec. and Bacillus spec.

7. The pharmaceutical composition for use of 4 or 5, wherein the pathogenic microbial taxa comprises Staphylococcus aureus, Staphylococcus epidermidis, Pseudomonas aeruginosa, Enterococcus faecalis/faecium, E. coli, Klebsiella pneumoniae, Candida albicans, Microsporum canis, Acinetobacter baumanii, Staphylococcus intermedius and Staphylococcus pseudointermedius.

8. The pharmaceutical composition for use of 1 to 7, further comprising urea, glycolic acid, glyoxylic acid, glycerol, pentylene glycol, lactic acid, ascorbic acid, pyroglutamic acid, citric acid, tartaric acid, fumaric acid, succinic acid, malic acid, mandelic acid, aloe vera, rosa gallica, hyaluronic acid, salicylic acid, gallic acid, cellulose and derivatives thereof, pectin and derivatives thereof, gummi arabicum, dextrines, cyclodextrines, xanthan gum, thiocyanate, amino acids, sorbic acid, sodium chloride or combinations thereof.

9. The pharmaceutical composition for use of 1 to 7, further comprising glycolic acid and/or lactic acid.

10. The pharmaceutical composition for use of 9, wherein the composition comprises

0.01 to 4.0% (w/w) chitosan,

0.05 to 2.5% (w/w) glycolic acid,

0.05 to 2.5% (w/w) lactic acid,

based on the total weight of the liquid cosmetic composition.

11. The pharmaceutical composition for use of 8 to 10, wherein the composition comprises a disinfectant.

12. The pharmaceutical composition for use of 11, wherein the disinfectant comprises an alcohol, aldehyde, iodine, chlorine, quarternary ammonia compound, peroxide, amphotenside, phenol, alkylamine, acid and/or base.

13. The pharmaceutical composition for use of 1 to 12, wherein the ectodermal tissue is the skin.

14. The pharmaceutical composition for use of 1 to 12, wherein the ectodermal tissue is the epidermis.

15. The pharmaceutical composition for use of 14, wherein the epidermis is damaged epidermis.

16. The pharmaceutical composition for use of 15, wherein the damage comprises an acute or chronic wound.

17. The pharmaceutical composition for use of 1 to 16, wherein the subject's ectodermal tissue, skin, epidermis, damaged epidermis, acute or chronic wound has previously been sterilized or disinfected.

18. The pharmaceutical composition for use of 1 to 17, wherein the dysbiosis is idiopathic (e.g., the subject has no substantially observable cause of a dysbiosis).

19. The pharmaceutical composition for use of 1 to 17, wherein the dysbiosis is associated with a disease, disorder, or condition in the subject.

20. The pharmaceutical composition for use of 19, wherein the disease, disorder, or condition comprises an infectious disease, an inflammatory disease, a metabolic disease, an autoimmune disease or a cancer.

21. The pharmaceutical composition for use of 20, wherein the infectious disease is a viral, microbial and/or fungal skin infection.

22. The pharmaceutical composition for use of 20, wherein the autoimmune disease is Crohn's disease, psoriasis, allergy, asthma, urticaria or atopic dermatitis.

23. The pharmaceutical composition for use of 1 to 17, wherein the dysbiosis is associated with or a consequence of immunodeficiency or low immunity, immunosuppression, burns or radiation of the skin, stress of the skin, the body or areas thereof.

24. The pharmaceutical composition for use of 23, wherein the stress of the skin comprises stress due to prostheses, endoprostheses, orthoses, exoskeletons, plasters, compression bandages, stockings, bandages, latex protection, masks for ventilation, long lying or sitting or wound dressings.

25. The pharmaceutical composition for use of 1 to 17, wherein the dysbiosis is associated with or a consequence of the administration of medicaments.

26. The pharmaceutical composition for use of 25, wherein the medicament comprises antibiotics, chemotherapeutics, antibodies, cytokines, cell-therapeutics, therapeutic nucleic acids or immunesuppressants.

27. The pharmaceutical composition for use of 23, wherein the radiation is ultraviolet radiation, gamma radiation, electron radiation, X-rays or sun rays.

28. Kit comprising chitosan in a liquid dosage form according to any of the preceding claims, further comprising a disinfectant, preferentially selected from the group comprising an alcohol, aldehyde, iodine, chlorine, quarternary ammonia compound, peroxide, amphotenside, phenol, alkylamine, acid and/or base.

### Definitions

As used herein, the term “dissolved”, “dissolution” and the like in the context of a polymer is meant to refer to solution of polymer in an aqueous environment without molecular weight decrease in polymer chain length. It is thus to be distinguished from “degradation”, which is the process of molecular weight decrease due to depolymerization of a polymer.

As used herein, the term “membrane” refers to a film-like selectively permeable barrier that is applied onto the skin and may be of cosmetic or medical nature. The membrane is however removable from the skin as a whole and may thus also exist isolated from the skin. In the context of the present invention, the term “membrane” also indicates that the thickness of the membrane is preferably very low, namely no more than 50 μm. In contrast, the more generic term of a “film” or “cosmetic film” also includes much thicker layers of a topically applied cosmetic product applied to the skin. In the context of chitosan, this may mean that a “cosmetic film” may be realized where the chitosan has not precipitated and the cosmetic composition is merely applied as a thick layer, e.g. of a gel or cream, while in a respective “membrane”, the chitosan has precipitated to form a membrane, optionally as a separate layer within the cosmetic film. A “cosmetic membrane” is not intended for the treatment of any specific disease. A “medical membrane” is intended for the treatment of a specific disease.

As used herein, the term “% degree acetylation” or “% DA”, as in “20% degree acetylation”, refers to the number of —NN₂ groups that are acetylated over the number of all —NH₂ present within a polymer. For example, if 20 —NH₂ groups are acetylated in a polymer and the polymer has 100 —NH₂ groups in total, including the 20 acetylated —NH₂ groups then the polymer has a 20% degree of acetylation.

As used herein, the term “area”, as in “area of a subject's skin” refers to the top surface area of the subject's skin. The subject is a human or an animal, preferably a human.

The term “ectodermal tissue” refers to any ectodermal derived tissue. Thus, surface/external ectodermal tissue includes but is not limited to the following cells, structures and tissues, i.e., pluristratified epidermis, epidermis of the skin, including glands, hair and nails (keratinocytes), epithelium of the mouth and nasal cavity, as well as salivary glands, enamel, epithelial of pineal and pituitary glands, lens and cornea and apical ectodermal ridge.

As used herein the term “skin” refers to the body's, preferably the human body's, outer layer and the term “skin” may comprise skin with and/or without hair. The skin is thus any type of skin, e.g. of the face, neck, mouth, throat, mucous membranes, chest, arms, legs, and covering other body parts, which are hair free or may comprise at least some body hair, such as hair on the arms or legs. However, in a preferred embodiment, the term “skin” also additionally comprises scalp. In another embodiment, the term “skin” does not comprises scalp. The skin may be the skin of a human or an animal and preferably a human.

The term “epidermis” refers to the outermost layer of skin, provides a waterproof barrier and creates the skin tone. The dermis, beneath the epidermis, contains tough connective tissue, hair follicles, and sweat glands. The epidermal cell is defined as an epithelial cell which constitutes the epidermis. The epidermis comprises a keratinized stratified squamous epithelium comprising, from the dermis towards the outer surface, stratum basale, stratum spinosum, stratum granulosum, stratum lucidum and stratum corneum.

As used herein, the term “cosmetic agent” refers to any substance intended to be placed in contact with the various external parts of the human body, such as epidermis, hair system, nails, lips and external genial organs, or with the teeth and the mucous membranes of the oral cavity for cleaning them, perfuming them, changing their appearance and/or correcting body odours and/or protecting them or keeping them in good condition. Preferably, the cosmetic agent keeps the skin in good condition. Examples of cosmetic agents nonexclusively include emollients, humectants, colorants, pigments, fragrances, moisturizers, viscosity modifiers and any other cosmetic forming agent. A cosmetic agent is not intended for the treatment of any specific disease.

As used herein, the term “cosmetic composition” refers to a preparation comprising at least one cosmetic agent intended to be placed in contact with the various external parts of the human body, such as epidermis, hair system, nails, lips and external genial organs, or with the teeth and the mucous membranes of the oral cavity for cleaning them, perfuming them, changing their appearance and/or correcting body odors and/or protecting them or keeping them in good condition. Preferably, the cosmetic agent keeps the skin in good condition. A cosmetic agent is not intended for the treatment of any specific disease.

As used herein, the term “liquid” refers to one of the four fundamental states of matter: liquid, solid, gas and plasma. The term “liquid” also comprises “semi-liquid states” such as a gel, a suspension, a dispersion, a foam, an emulsion or a gel.

As used herein, the term “pH of a cosmetic membrane” means the pH that can be measured when applying a drop of deionized water on top of the cosmetic membrane and the pH is then measured on the wetted skin with a flat tip pH electrode.

As used herein, the term “thickness” in the context of the thickness of a cosmetic membrane means the thickness of the layer that is formed on top of a substrate, e.g. the skin, by the cosmetic membrane. The thickness of the cosmetic membrane may be calculated based on the volume and chitosan concentration of the liquid cosmetic composition, e.g. 0.1 to 1 μl having a concentration of 0.5% to 10% (w/w) based on the total weight of the liquid cosmetic composition, and the area of the subject's skin that it is applied to, e.g. 5 cm². Further, the thickness of the membrane is calculated based on the density of the membrane, which can be assumed to be about 1.5 g/cm³. For example, for a skin area of 25 cm² and an application volume of 1 μl, a thickness of 27 nm can be calculated for a 10% (w/w) chitosan containing liquid cosmetic composition. Assuming that the membrane still comprises about 50% (w/w) water molecules, the thickness may double to about 54 nm.

Alternatively, the thickness of the isolated cosmetic membrane could be determined using an ellipsometer.

As used herein, the term “wetting agent” refers to a surfactant, i.e. a substance that increases the spreading properties of a liquid by lowering its surface tension, i.e. the tendency of its molecules to adhere to each other.

As used herein, the term “skin care application” refers to any kind of cosmetic treatment that keeps the skin in good condition or improves the condition of stressed or irritated skin, i.e. the skin care application does not improve the condition of a specific pathological state such as psoriasis or other commonly known skin diseases.

As used herein, the term “sprayable liquid” refers to a liquid which can be applied to the skin as a spray from any type of commonly used cosmetic spray dispensers.

As used herein “topically applying” means directly laying on or spreading on outer skin, e.g. by use of hands or an applicator such as a wipe, puff, roll, foam or spray. As used herein, the term “about” as e.g. in “about 4.5 to 5.5” means that the value recited immediately after the “about” also comprises minor deviations from the exact numeric value, e.g. due to measuring errors.

As used herein, the term “skin scar tissue” refers to scar tissue that has replaced skin tissue, e.g. after the respective skin had been wounded or after surgery where the skin was opened and the respective area was replaced by scar tissue.

As used herein, the term “skin scar tissue” refers to a type of injury in which skin is torn, cut, or punctured (an open wound), or where blunt force trauma causes a contusion (a closed wound). A wound often damages the epidermis of the skin. Wounds may also comprise closed wounds, which are in the process of healing.

As used herein, the term “microbiome” refers to the genetic content of the communities of microbes that live in and on a subject (e.g. a human subject), both sustainably and transiently, including eukaryotes, archaea, bacteria, and viruses (including bacterial viruses (e.g., phage)), wherein “genetic content” includes genomic DNA, RNA such as ribosomal RNA and messenger RNA, the epigenome, plasmids, and all other types of genetic information. In some embodiments, microbiome specifically refers to genetic content of the communities of microorganisms in a niche.

“Microbiota” as used herein refers to the community of microorganisms that occur (sustainably or transiently) in and on a subject (e.g. a human subject), including eukaryotes, archaea, bacteria, and viruses (including bacterial viruses, e.g. phage). In some embodiments, microbiota specifically refers to the microbial community in a niche.

“Microflora” as used herein is synonymous with “microbiome” and “microbiota”. The terms “microbiome” and “microbiota” essentially differ in their readout. Whereas “microbiome” is analysed by genetic methods such as PCR, the “microbiota” is analysed by microbiological methods such as growing the respective microbes on an agar plate.

As used herein, “colonization” of a host organism refers to the non-transitory residence of a bacterium or other microbial organism in a niche.

As used herein, the term “abundance” as it relates to a microbial taxa refers to the presence of one microbial taxa as compared to another microbial taxa in a defined microbial niche, such as the GI tract, or in the entire host organism (e.g. a human or a laboratory animal model of disease). The microbial taxa may be a bacterial taxa.

As used herein, the term “taxa,” or “taxon,”, generally refers to a group of microbes adjudged to be a unit. Microbes may be classified into taxa by a host of different types of characteristics.

As used herein, the term “microbe” or “microbial”, generally refers to a living thing that is too small to be seen with the naked eye. Exemplary microbes include but are not limited to bacteria, archaea, fungi, protists, viruses and microscopic animals.

As used herein, a “dysbiosis” refers to the state of the microbiota under conditions of host disease, predisposition to host disease, or another unwanted condition or symptom of the host. In an embodiment, dysbiosis refers to the state of the microbiota under conditions of disease. Dysbiosis can be contrasted with eubiosis, which refers to the state of the microbiota under healthy conditions of the host. The state of the microbiota may include the characteristics relating to either the structure or function of the microbiota. In an embodiment, a dysbiosis includes an imbalance in the state of the microbiota, wherein the normal diversity or relative abundance of a microbial taxa is affected, e.g., relative to a second bacterial taxa or relative to the abundance of said taxa under conditions of health. In an embodiment, a dysbiosis comprises an imbalance in the function of the microbiota, e.g., a change in level of gene expression, level of a gene product, or metabolic output (e.g., an immune function such as immune surveillance or the inflammation response). In some embodiments, a dysbiosis is an undesired, e.g., unhealthy, state associated with unwanted symptoms in the host and that no longer promotes health.

“Modulate the microbiota” or “modulating the microbiota” as used herein refers to changing the state of the microbiota. Changing the state of the microbiota may include changing the structure and/or function of the microbiota. A change in the structure of the microbiota is, e.g., a change in the relative composition of a taxa, e.g., in one or more regions of the ectodermal tissue, skin or epidermis. In an embodiment, a change in the structure of the microbiota comprises a change in the abundance of a taxa, e.g., relative to another taxa or relative to what would be observed in the absence of the modulation. Modulation of the microbiota may also, or in addition, include a change in a function of the microbiota, such as a change in microbiota gene expression, level of a gene product (e.g., RNA or protein), or metabolic output of the microbiota. Functions of the microbiota may also include host pathogen protection, host nutrition, host metabolism and host immune modulation. Modulation of the structure or function of the microbiota may additionally induce a change in one or more functional pathway of the host (e.g., a change in gene expression, level of a gene product, and/or metabolic output of a host cell or host process) as a result of a change in the microbiota or its function.

As used herein, the term “pathogenic” (e.g. “pathogenic bacteria”) refers to a substance, microorganism or condition that has the capability to cause a disease. In certain contexts, pathogens also include microbes (e.g. bacteria) that are associated with a disease or condition but for which a causative relationship (e.g., a direct causative relationship) has not been established or has yet to be established.

The term “phenotype” refers to a set of observable characteristics of an individual entity. For example, an individual subject may have a phenotype of “healthy” or “diseased.” A phenotype may describe the state of an entity, wherein all entities within a phenotype share the same set of characteristics that describe the phenotype. The phenotype of an individual results in part, or in whole, from the interaction of the entities genome and/or microbiome with the environment.

As used herein, the term “subject” or “patient” generally refers to any human or animal subject. A human does not refer to a particular age or gender. Subjects may include pregnant women. Subjects may include a newborn (a preterm newborn, a full term newborn), an infant up to one year of age, young children (e.g., 1 yr to 12 yrs), teenagers, (e.g., 13-19 yrs), adults (e.g., 20-64 yrs), and elderly adults (65 yrs and older). A subject does include animals such as pets, agricultural animals, e.g., farm animals or livestock, e.g., cattle, horses, sheep, swine, chickens, etc. as well as wild animals. In general, a subject comprises a host and its corresponding microbiota.

The terms “treating” and “treatment” as used herein refer to the administration of an agent or composition to a subject (e.g., a symptomatic subject afflicted with an adverse condition, disorder, or disease) so as to affect a reduction in severity and/or frequency of a symptom, eliminate a symptom and/or its underlying cause, and/or facilitate improvement or remediation of damage, and/or preventing an adverse condition, disorder, or disease in an asymptomatic subject who is susceptible to a particular adverse condition, disorder, or disease, or who is suspected of developing or at risk of developing the condition, disorder, or disease.

Finally, the present invention still relates to the following embodiments:

1. Liquid cosmetic composition comprising

a) chitosan or a salt thereof, wherein the degree of acetylation of the chitosan is 20% or less, and

b) at least one further cosmetic agent;

wherein the pH of the liquid cosmetic composition is from about 4.0 to about 6.5.

2. Liquid cosmetic composition according to 1, wherein the at least one further cosmetic agent is selected from the group comprising urea, glycolic acid, glyoxylic acid, glycerol, pentylene glycol, lactic acid, ascorbic acid, pyroglutamic acid, citric acid, tartaric acid, fumaric acid, succinic acid, malic acid, mandelic acid, aloe vera, rosa gallica, hyaluronic acid, salicylic acid, gallic acid, cellulose and derivatives thereof, pectin and derivatives thereof, gummi arabicum, dextrines, cyclodextrines, xanthan gum, thiocyanate, amino acids, sorbic acid, sodium chloride or combinations thereof.

3. Liquid cosmetic composition according to 2, wherein the at least one further cosmetic agent comprises glycolic acid and lactic acid.

4. Liquid cosmetic composition according to any one of 1 to 3, wherein the chitosan is de-acetylated in steps.

5. Liquid cosmetic composition according to any one of 1 to 4, wherein the degree of acetylation of the chitosan is 15% or less, more preferably 10% or less, even more preferably 5% or less, or even more preferably 2.5% or less.

6. Liquid cosmetic composition according to any one of 1 to 5, wherein the concentration of the chitosan is from at least 0.1% to 15% (w/w) based on the total weight of the liquid cosmetic composition.

7. Liquid cosmetic composition according to any one of 1 to 5, wherein the composition comprises

0.05% to 15% (w/w) chitosan,

0.0% to 9% (w/w) glycolic acid,

0.0% to 9% (w/w) lactic acid,

based on the total weight of the liquid cosmetic composition.

8. Liquid cosmetic composition according to any one of 1 to 5, wherein the composition comprises

0.20 to 4.0% (w/w) chitosan,

0.0 to 2.5% (w/w) glycolic acid,

0.0 to 2.5% (w/w) lactic acid,

based on the total weight of the liquid cosmetic composition.

9. Liquid cosmetic composition according to any one of 1 to 8, wherein the liquid cosmetic composition further comprises an emulsifier, surfactant or wetting agent.

10. Liquid cosmetic composition according to 9, wherein the emulsifier, surfactant or wetting agent is selected from the group comprising polysorbates, alkyl amido betaines, alcohol polyglucosides or combinations thereof.

11. Liquid cosmetic composition according to any one of 1 to 10, wherein the liquid cosmetic composition further comprises a preservative.

12. Liquid cosmetic composition according to 11, wherein the preservative is sorbic acid as free acid or a salt thereof.

13. Liquid cosmetic composition according to any one of 1 to 10, wherein the liquid cosmetic composition comprises no preservative.

14. Liquid cosmetic composition according to any one of 1 to 13, wherein the liquid cosmetic composition is in the form of an emulsion, dispersion, suspension, serum, gel, a solution, a sprayable liquid, aerosol or foam.

15. Use of the liquid cosmetic composition according to any one of 1 to 14 for skin care applications, wherein the liquid cosmetic composition is applied topically on an area of a subject's skin to form a cosmetic membrane.

16. Use according to 15, wherein the skin care application is a care of abrasions, care of cuts, care of pimples, care of blisters, care of stings, care of burns, anti-ageing application, care of irritated skin, care of radiated skin, care of laser treatments, care of plasma treatments, care of tattoos and removal of tattoos, care or prevention of skin peeling, care or prevention of radiation induced skin changes, care or prevention of formation of skin scar tissue, prevention of dry skin, prevention of fatty skin, prevention of cracking of skin, prevention of stretch marks, reduction of wrinkling of the skin, protection of thin skin or a skin cleansing application or prevention of accompanying symptoms thereof.

17. Use according to 16, wherein the skin care application is the prevention of accompanying symptoms, wherein the accompanying symptom is selected from the group consisting of surface pain, itching, wet feeling, smell, roughness, crackiness, fissures, dryness, ugliness, peeling or other unpleasant stress symptoms or combinations thereof.

18. Use according to 15, 16 or 17, wherein the cosmetic membrane forms a stable support for conventional make-up which is applied following the cosmetic membrane's formation with no disturbing optical impact.

19. Use according to any one of 15 to 18, wherein the cosmetic membrane has a pH of from about 4.0 to about 6.

20. Use according to any one of 15 to 19, wherein the liquid cosmetic composition comprises

0.20 to 4.0% (w/w) chitosan,

0.0 to 2.5% (w/w) glycolic acid,

0.0 to 2.5% (w/w) lactic acid,

based on the total weight of the liquid cosmetic composition.

### EXAMPLES

### Example 1 Comparison of pH Before and After Cosmetic Treatment with Chitosan Membrane-Forming Compositions

Solutions A and B with low-acetylated chitosan with a degree of acetylation of 2% were prepared as follows:

Solution A (Ch-Lac/Gly)

| | |
|---|---|
| Chitosan | 3.00% |
| Glycolic Acid | 0.74% |
| Lactic Acid | 0.88% |
| Potassium sorbate | 0.10% |
| Glycerol | 1.00% |
| Polysorbate 20 | 0.25% |

pH 4.8

Solution B (Ch-Ac)

| | |
|---|---|
| Chitosan | 3.00% |
| Acetic Acid | 1.23% |
| Potassium sorbate | 0.10% |
| Glycerol | 1.00% |
| Polysorbate 20 | 0.25% |

pH 5.0

All of the above percentages relate to w/w. Lactic acid and glycolic acid are commonly known cosmetic moisturizing agents. Potassium sorbate is a commonly known preservative and glycerol a further commonly known humectant of cosmetic compositions. Polysorbate 20 is a commonly known wetting agent.

As a reference, Solution C was prepared without Chitosan.

Solution C (Na-Lac/Gly)

| | |
|---|---|
| Glycolic Acid | 0.74% |
| Lactic Acid | 0.88% |

Adjusted to pH 4.9 with NaOH

All of the above percentages relate to w/w. All solutions A, B and C provide about the same pH value. The low-acetylated chitosan with an acetylation degree of 2% was obtained according to [0065] of EP 2 473 202 B1 wherein the parameters of the method have been adjusted in order to achieve an acetylation degree of 2%.

For further experiments, solutions A, B and C were diluted as described in Table 1 to 3:

TABLE 1
Dilution of Solution A

| Dilution name | Solution A | Glycerol (1%) in water | Chitosan conc. |
|---|---|---|---|
| A 1 | 0.33 g | 9.67 g | 0.1% |
| A 2 | 1.00 g | 9.00 g | 0.3% |
| A 3 | 1.67 g | 8.33 g | 0.5% |
| A 4 | 2.50 g | 7.50 g | 0.75% |
| A 5 | 3.33 g | 6.67 g | 1.0% |
| A 6 | 5.00 g | 5.00 g | 1.5% |
| A 7 | 7.50 g | 2.50 g | 2.25% |
| A 8 | 10.00 g | 0.00 g | 3.0% |

TABLE 2
Dilution of Solution B

| Dilution name | Solution B | Glycerol (1%) in water | Chitosan conc. |
|---|---|---|---|
| B 1 | 0.33 g | 9.67 g | 0.1% |
| B 2 | 1.00 g | 9.00 g | 0.3% |
| B 3 | 1.67 g | 8.33 g | 0.5% |
| B 4 | 2.50 g | 7.50 g | 0.75% |
| B 5 | 3.33 g | 6.67 g | 1.0% |
| B 6 | 5.00 g | 5.00 g | 1.5% |
| B 7 | 7.50 g | 2.50 g | 2.25% |
| B 8 | 10.00 g | 0.00 g | 3.0% |

TABLE 3
Dilution of Solution C

| Dilution name | Solution C | Water | Chitosan conc. |
|---|---|---|---|
| C 1 | 0.33 g | 9.67 g | 0% |
| C 2 | 1.00 g | 9.00 g | 0% |
| C 3 | 1.67 g | 8.33 g | 0% |
| C 4 | 2.50 g | 7.50 g | 0% |
| C 5 | 3.33 g | 6.67 g | 0% |
| C 6 | 5.00 g | 5.00 g | 0% |
| C 7 | 7.50 g | 2.50 g | 0% |
| C 8 | 10.00 g | 0.00 g | 0% |

For measuring the pH of the skin, the respective area of the skin became wetted with a 50 μl drop of deionized water and the pH was determined using a calibrated flat tip pH-electrode (pH electrode HI 14142 of Hanna Instruments GmbH) by contacting the pH-electrode with the wetted skin.

The natural pH of the skin of the test subject was determined to be pH 4.6 and was raised above pH 6.0 by washing with curd soap.

Further, the respective dilutions of A, B and C were applied to the skin and distributed by wiping with the pipette tip over an area of 5 cm². After 3 minutes, the skin area was dry. It was assumed that a membrane with a thickness between 1 μm and 6 μm had formed, at least for dilutions of A and B. A 50 μl drop of deionized water was positioned on the respective area and the pH-electrode got contacted with the drop.

The respective change in pH is summarized in Table 4 below:

TABLE 4
pH change after application of dilutions 1 to 8 of solutions A to C

| | ΔpH Na- | ΔpH Ch-Ac | ΔpH Ch- |
|---|---|---|---|
| Dilution | Lac/Gly (,,C”) | (,,B) | Lac/Gly (,,A”) |
| 1 | −0.03 | −0.04 | −0.21 |
| 2 | −0.01 | −0.11 | −0.56 |
| 3 | −0.11 | −0.37 | −0.68 |
| 4 | −0.09 | −0.37 | −0.86 |
| 5 | −0.13 | −0.47 | −0.88 |
| 6 | −0.23 | −0.57 | −0.98 |
| 7 | −0.19 | −0.58 | −1.08 |
| 8 | −0.22 | −0.54 | −1.08 |

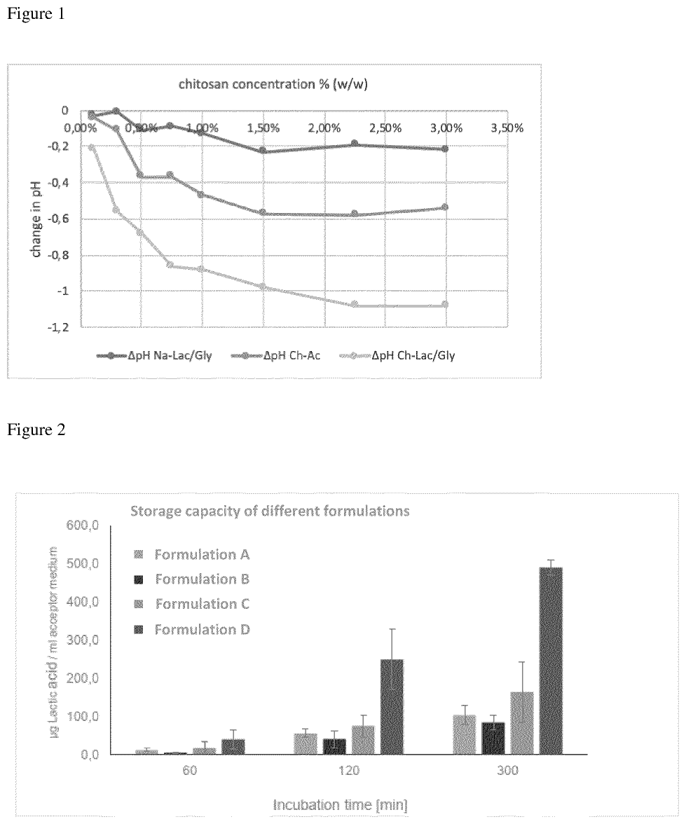

The results are also summarized in FIG. 1.

Chitosan-containing solutions forming a membrane show a significant decrease of pH towards the natural pH of the skin of below pH 5.0 above concentrations of 0.3% chitosan. A minimum pH was reached when using solutions A and B comprising between 1.0% and 3.0% chitosan.

The effect is more pronounced with solution A than with solution B.

The effect is dramatically smaller when the same acids of solution A are used without chitosan. Without being bound to theory, it appears that acids of solution C enter directly into the skin and do not remain on the surface of the skin.

### Example 2 Skin PAMPA with Chitosan Membrane-Forming Compositions

Skin PAMPA (Parallel Artificial Membrane Permeability Assay) was conducted to test the hypothesis that the release of active substances from the chitosan membrane-forming compositions according to the invention are modified or delayed compared to other formulations containing a different polymer or no polymer at all. The permeation of lactic acid was measured using a Skin PAMPA sandwich consisting of two 96-well plates with one plate formed to sit precisely under the plate that contains a porous lipid-impregnated filter. The wells of the bottom plate were filled with acceptor solution and the wells of the top plate were filled with the four different test formulations A-D. The plates were then piled up and incubated. The Skin PAMPA model has been used for the evaluation of semisolid formulations and was found to correlate well with ex vivo permeation studies (Sinko et al., 2012; Luo et al., 2016). The release profiles from the four test formulations A-D were measured at three different timepoints using lactic acid as a marker molecule to determine the storage potential of the different test formulations.

The following formulations A-D were tested.

Formulations A-D

| | Formu- | Formu- | Formu- | Formu- |
|---|---|---|---|---|
| | lation A | lation B | lation C | lation D |
| Polymer | 3.00% | 3.00% | 3.00% | — |
| | (Ac 2%) | (Ac 15%) | Natrosol ™ | |
| | chitosan | chitosan | 250 HX | |
| Ascorbic Acid | 0.01% | 0.01% | 0.01% | 0.01% |
| Potassium | 0.25% | 0.25% | 0.25% | 0.25% |
| sorbate | | | | |
| Glycolic acid | 0.67% | 0.98% | 0.98% | 0.98% |
| Lactic acid | 0.98% | 1.09% | 1.09% | 1.09% |
| Potassium | 0.10% | 0.25% | 0.25% | 0.25% |
| sorbate (10%) | | | | |
| Polysorbate 20 | 0.75% | 0.75% | 0.75% | 0.75% |
| Glycerol | 5.00% | 5.81% | 5.81% | 5.81% |
| Urea | 5.00% | 5.00% | 5.00% | 5.00% |
| Aqua | add. 100% | add. 100% | add. 100% | add. 100% |

All % in the above table are w/w. The formulations were prepared by mixing the 10% potassium sorbate component with a solution of lactic acid, glycolic acid, the polymer (or no polymer for formulation D) and water and stirring for 4 hours. Then the glycerol component was added including urea and polysorbate 20 and the mixture was mixed for 1 minute. In a last step, potassium sorbate and ascorbic acid were added and the solution was stirred. The pH was determined and adjusted to pH 5±0.2 using NaOH.

Based on a theoretical assessment taking into account chemical compatibility, pH and sink conditions, an acceptor phase composed of 20 mM phosphate buffer, pH 5.0 was selected. To evaluate suitable timepoints for the current study, a Skin PAMPA experiment with Formulation A in 20 mM phosphate buffer, pH 5 was analyzed at 30, 90 and 300 min. 25 μl of each sample was applied into the wells of the donor compartment. At all three timepoints, detectable amounts of lactic acid were observed in the acceptor compartment. However, the 30 min values of lactic acid released from the test formulation were close to the detection limit of the initial analytical method. Thus, in further studies, the assay timepoints were set to 60, 120 and 300 min and the analytical method was adjusted to be suitable to determine a lower concentrations of lactic acid. To obtain an initial impression of the evaporation time of the tested formulations, 10 μl and 25 μl of Formulation A and C were applied on a coated paper card and film-forming was monitored by visual inspection of the cards. As expected, film-forming occurred slightly faster with 10 μl sample but with both application volumes and formulations, a film was formed within one hour. The evaporation behavior of the different formulations in the wells of the Skin PAMPA might differ due to the hydrated state of the artificial membrane. The assay was conducted without a lid to facilitate the formation of films from polymer-containing formulations. Based on the preliminary assessment a final sample volume of 10 μl was chosen for the main experiment. Due to the high viscosity of the formulations, a pipette relying on positive-displacement technology was used to transfer the solutions into the wells. The pH of all formulations was recorded after preparation and adjusted to pH 5 with 30% sodium hydroxide. Sodium hydroxide was poured slowly into the formulations to avoid chemical reactions in the area where the base enters the formulation. At the end of the experiment, a shimmery film was observed in all wells with polymer-containing formulations whereas the wells with the formulation that does not contain a polymer, i.e. Formulation D, appeared to be empty (possibly due to evaporation of water). Most of the films appeared to be dry as indicated by the absence of sucked up formulation when touched with the end of a tip.

Lactic acid was measured in samples using analytical HPLC by transfer of 180 μl of each Skin PAMPA sample into a HPLC vial containing a 300 μl glass HPLC tube. 20 μl 8.5% phosphoric acid were added and mixed well. Samples were measured against a reference standard.

Analytical Conditions

Equipment: Agilent HP 1100 connected to Waters Empower 3 SR3

Column: Waters Atlantis® T3 3.0×150 mm, 3 μm

Column temp.: 30° C.

Mobile phase A: 0.1% phosphoric acid in water

Mobile phase B: Acetonitrile

Flow: 0.23 ml/min

Injection vol.: 12 μl

Wavelength: 210 nm

Gradient Table

| Time [min] | % A | % B |
|---|---|---|
| 0.00 | 100.0 | 0.0 |
| 4.00 | 100.0 | 0.0 |
| 4.01 | 10.0 | 90.0 |
| 5.00 | 10.0 | 90.0 |
| 5.01 | 100.0 | 0.0 |
| 20.0 | 100.0 | 0.0 |

Retention time Lactic acid approx. 7.2 min.

Evaluation: linear, 1/x-weighting

Analytical acceptance criteria:

% Deviation of recalculated calibration samples: ≤15%

Correlation coefficient r: ≥0.999

% Deviation of quality control samples: ≤15%

Results

Permeation of lactic acid from four formulations A-D was evaluated with Skin PAMPA at three different timepoints. It is hypothesized that the polymers influence the release rate of the selected marker. Formulation D without chitosan (and without any other polymer) was used as reference sample. In this experiment, the formulation D without any polymer, showed significantly higher values of lactic acid. Formulation C containing Natrosol™ 250HX showed higher values of lactic acid than the two formulations according to the invention containing chitosan, formulations A and B. Hence, there is a trend for a lower permeation of the membrane with chitosan containing formulations. It can be assumed that in formulations containing the cationic polymer chitosan, the release of lactic acid, which is predominantly negatively charged at the formulation pH of 5, is delayed due to interactions versus polymer-free formulation or neutral polymers. In addition, the speed of diffusion of dissolved actives can also be influenced by the viscosity of the formulation—which, however, was not subject of this study. The ranking of the different formulations (D>C>A>B) was the same at all timepoints tested but with an increase in permeated amounts with regard to respective earlier timepoints. Between 60 and 120 min (within 1 hour) a steep increase in permeation was observed for all samples whereas within the next 180 min only a moderate increase was observed. Each timepoint was conducted on a separate Skin PAMPA plate indicating the reproducibility of the assay. The standard deviations in the current experiment are comparable to standard deviations described in the literature (Sinko et al., 2014).

### Example 3 Determination of the Influence of Chitosan Membrane-Forming Compositions on Organisms of the Human Core Skin Microbiome

Although the microbiome varies from human to human, the core microbiome comprises few major microorganisms which inter alia comprise Staphylococcus epidermidis, Staphylococcus mitis, Staphylococcus capitis, Corynebacterium specs., Propionibacterium acnes, Malassezia pachydermatis and Streptococcus spec.,

For the present experiment, the above microbiome organisms are seeded out on an agar plate. Afterwards the organisms are embedded in a special microbiome agar matrix to simulate the epidermis layers. The surface is then contaminated with Staphylococcus aureus as a typical pathogenic organism, which does not belong to a normal human skin microbiome. In the next step the test formulation is applied onto small areas on the surface and the test setup is incubated at 37° C. over night. After incubation, growth of the matrix-embedded microbiome organisms and superficial Staphylococcus aureus is used for the assessment of an influence of the test formulation on the microbiome organisms. If the applied test formulation is able to reduce or inhibit the growth of Staphylococcus aureus while maintaining or promoting the growth of the underlying microbiome organisms, the test formulation demonstrates good surface protection properties without affecting the skin microbiome. If the applied product also causes a growth inhibition of the microbiome organisms, the product is assessed to have negative influence on the human microbiome. A disinfectant agent (isopropanol:water 70:30) is used as negative control to inhibit growth of the pathogen as well as the microbiome organisms. Buffer solution (used for dilution, if applied; 0.9% (w/w) NaCl in water) is used as a negative control. The sample volume of test formulation is 20 μl. The inoculum microbiome contains 2.0×10⁶ CFU (colony forming units). The inoculum pathogen was set at 2.0×10⁶ CFU.

The following formulations E-F were tested:

| | Formulation E | Formulation G | Formulation F |
|---|---|---|---|
| Chitosan (Ac 2%) | 3.00% | 3.00% | 0.75% |
| Ascorbic Acid | | | |
| Potassium sorbate | 0.1% | 0.1% | 0.1% |
| Glycolic acid | 0.67% | — | — |
| Lactic acid | 0.98% | 1.86% | 0.47% |
| Polysorbate 20 | 0.25% | 0.19% | 1.00% |
| Glycerol | 5.00% | 5.00% | 1.00% |
| Urea | 1.00% | 5.00% | 1.00% |
| Aqua | add. 100% | add. 100% | add. 100% |

All % in the above table are w/w. Formulations E and G are intended as gel, while formulation F is intended as a spray.

Results

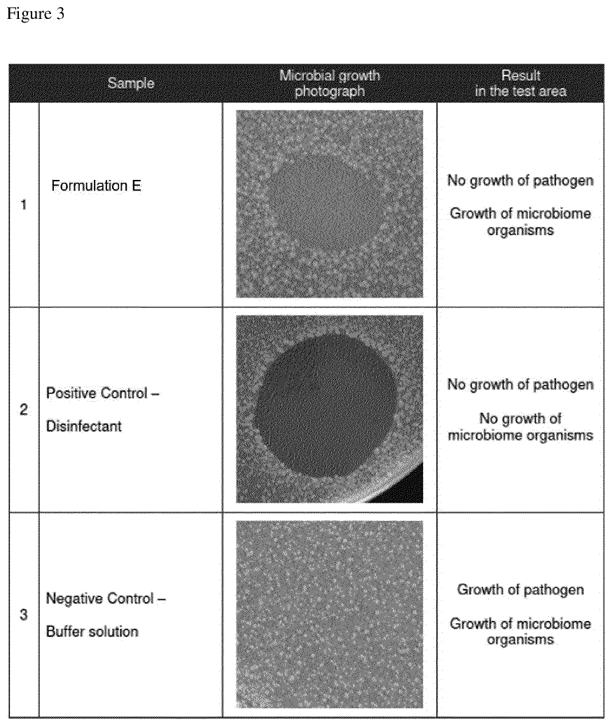

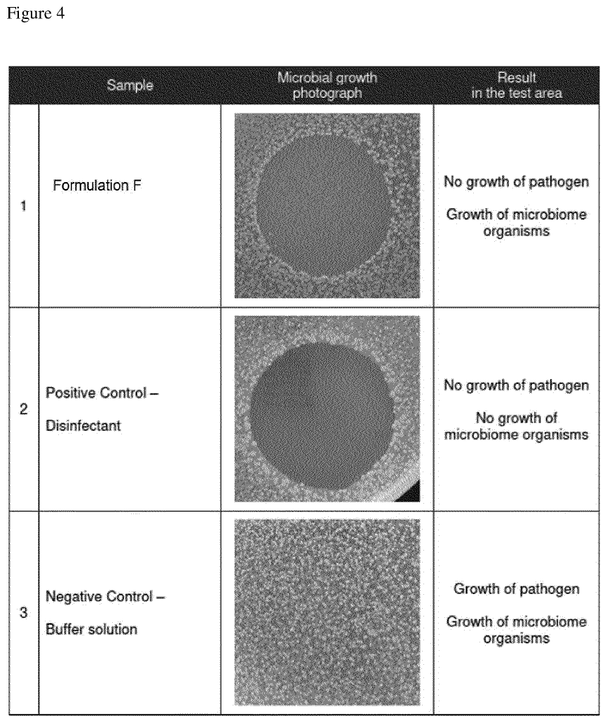

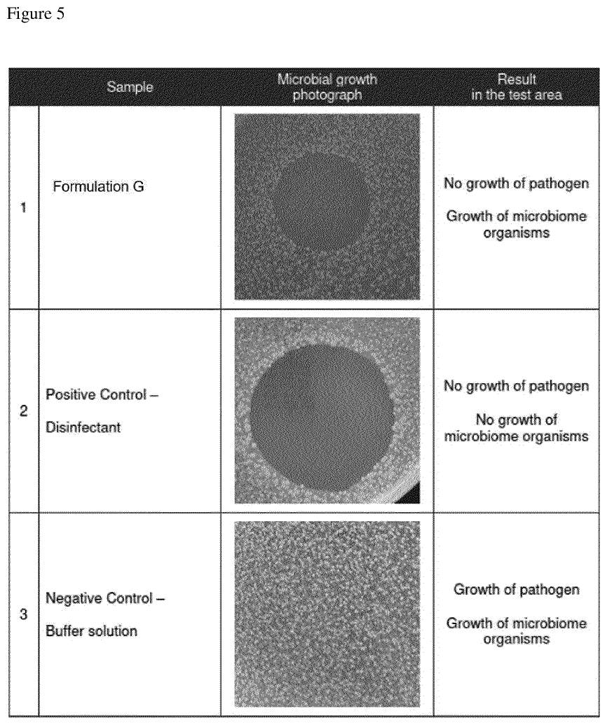

The test formulations E to F demonstrated a good surface protection by inhibiting growth of a pathogenic Staphylococcus aureus strain, whereas the underlying microbiome organisms were not affected by the surface treatment as can be seen from FIG. 3 (test formulation E), FIG. 4 (test formulation F) and FIG. 5 (test formulation G). The negative controls in FIGS. 3, 4 and 5 show growth on the surface, i.e. growth of Staphylococcus aureus. The positive controls in FIGS. 3, 4 and 5 show neither growth on the surface nor of the underlying layer of microbiome microorganisms.

### Example 4 Determination of Antimicrobial Efficacy Kinetics of a Liquid Composition Comprising Chitosan and Influence of Liquid Composition Comprising Chitosan and Disinfection on Repopulation of Human Skin

1. Test Method

The antimicrobial kinetics test is performed on the basis of the test method “Efficacy of antimicrobial preservation” of the European Pharmacopoeia. The test provides a visual and semi-quantitative overview of the antimicrobial efficacy of an antimicrobial sample compared to an untreated reference sample over a defined period of time.

2. Test Description

For this purpose, samples (approx. 3×3 cm-5×5 cm) or 0.5-1.0 ml of test solutions are contaminated with a defined number of bacteria and incubated for defined periods of time under standardized conditions. Time point t0 is used to demonstrate the initial contamination. At the end of the incubation period the vital microorganisms are recovered from the samples a dilution series in plated out an agar plates. The agar plates are incubated for 18-24 hrs at 37° C. and the number of colony forming units (CFU)s is counted.

3. Test Parameter for the Performed Test

The test germ corresponded to endogenous skin microflora. The sample material was test formulation E from Example 3 as well as disinfection solution (70% isopropanol). The sample size was approx. 50 cm² skin surface area and the sample volume 100 μl. Contact time was 0, 1 h, 2 h, 4 h, 6 h, after contact and 1 h after contact.

4. Comments on Test Samples, Performance and Results:

Rodac contact plates were used for sampling of microorganisms from human skin (upper arm) before and after treatment. The upper arm region was chosen to prevent contamination by touching and clothing. Disinfection was performed with 70% isopropanol.

5. References:

European Pharmacopoeia: 5.1.3. Efficacy of antimicrobial preservation.

6. Results

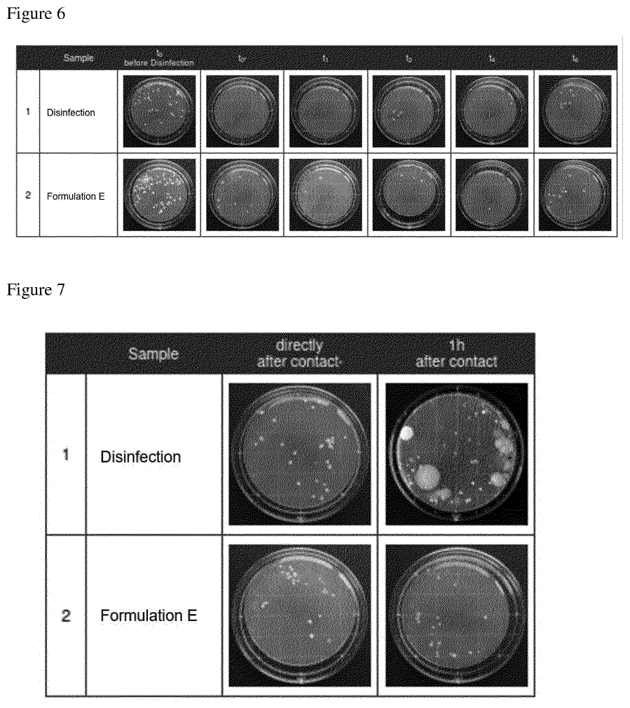

The results are depicted in FIG. 6. The test area in the upper arm were disinfected, air dried for 2 minutes. In case of “Formulation E”, Formulation E was applied on the test area and air dried. Time point t0 was taken after disinfection and application of Formulation E and drying. Bacteria were collected from the test areas by Rodac contact plates after the defined time points. The small white and yellowish colonies are typical Staphylococci species from the human skin microbiome. The yellow-orange colonies are Staphylococcus aureus, which does not belong to a normal skin microbiome. The Staphylococcus aureus was only found on the “Disinfection” test area, but not on the “Formulation E” test area.

Further results are depicted in FIG. 7. After the 6 h test period, the test areas were exposed to worn clothing for 10 minutes, resulting in a typical transfer of microorganism from clothing to the skin surface (time point directly after contact). 1 h after removal of clothing the bacteria were collected from the test areas aby Rodac contact plates. The sample “Formulation E” showed superficial microorganisms of a typical skin microflora. The sample “Disinfection” also showed pathogenic organisms (gram-negative bacteria=big colonies), which do not belong to the typical commensal and healthy skin microflora.

Overall, the results demonstrate that a chitosan containing composition according to the invention inhibits the growth of pathogenic microbial taxa such as Staphylococcus aureus. A chitosan containing composition according to the invention differentially promotes the growth of beneficial microbial taxa relative to pathogenic microbial taxa and appears to inhibit pathogenic microbes from colonizing previously disinfected areas. Disinfection first kills the microbes on the “test skin” but leaves an unprotected area, which is colonized by both beneficial and pathogenic microbes possibly leading to an unhealthy microbiome and even dysbiosis. Without being bound to a scientific theory, it may be assumed at present that said remarkable effects of Formulation E on the microbiome may inter alia be associated with the corresponding change in pH as seen in FIG. 1 for “ApH Ch-Lac/Gly”.

### Example 5 Determination of Antimicrobial Efficacy Kinetics of a Liquid Composition Comprising Chitosan and Influence of Liquid Composition Comprising Chitosan and Disinfection on Repopulation of Human Skin

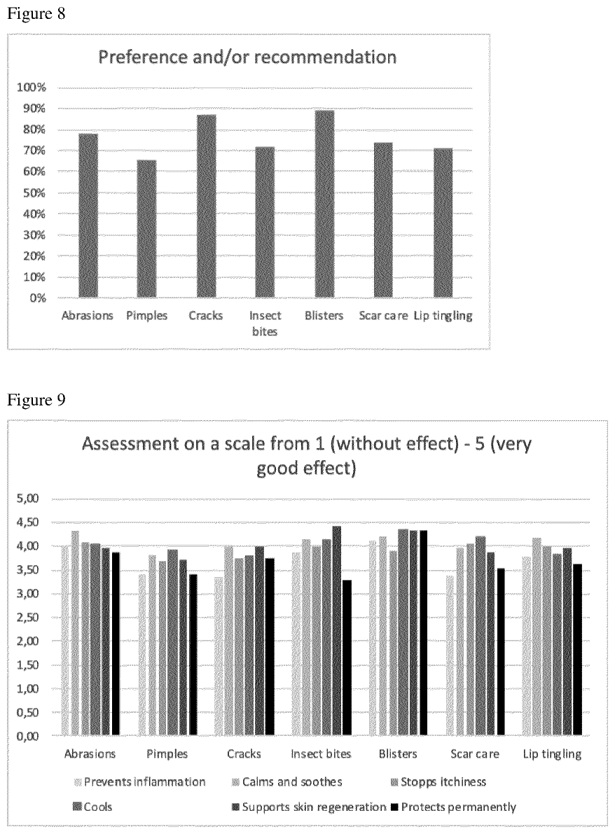

Effects of Formulation E (as described in Example 3) were further studied in a campagne including 300 voluntary testers. Each tester received a pack of Formulation E and was then able to provide feedback within two to four weeks via a corresponding website. The ratings were also provided on the website. There were no personal meetings with the test subjects. The results of the ratings as regards abrasions, pimples, cracks or fissures, insect bites, blisters, scar care and lip tingling are shown in FIGS. 8 and 9. Test formulation E has very good effects on all of the afore-mentioned applications according to the registered testers as can be derived from FIGS. 8 and 9. Formulation E would be preferred and/or recommended by the testers to a high degree for the treatment or care of abrasions, pimples, cracks or fissures, insect bites, blisters, scar care and lip tingling (FIG. 8). Furthermore, Formulation E shows very pronounced effects in the treatment of abrasions, pimples, cracks or fissures, insect bites, blisters, scar care and lip tingling according to the testers (FIG. 9).

1. Use of a liquid cosmetic composition comprising chitosan for differentially promoting the growth of the microbiota on a subject's ectodermal tissue.

2. The use according to claim 1, wherein the liquid cosmetic composition is applied topically to form a cosmetic membrane.

3. The use according to claim 2, wherein the formed membrane has a thickness of 0.001 nm to 50 μm, preferably 0.001 nm to 10 μm and more preferably from 0.001 nm to 1 μm.

4. The use according to claims 1 to 3, wherein the liquid cosmetic composition which forms a membrane differentially promotes the growth of one or more microbial taxa relative to another microbial taxa.

5. The use according to claim 4, wherein the liquid cosmetic composition which forms a membrane promotes the growth of one or more beneficial microbial taxa relative to one or more pathogenic microbial taxa.

6. The use according to claim 5, wherein the at least one beneficial taxa comprises Staphylococcus epidermidis, Staphylococcus mitis, Staphylococcus capitis, Corynebacterium specs., Propionibacterium acnes, Malassezia pachydermatis, Streptococcus spec., Streptococcus spec., Lactobacillus spec., Micrococcus spec. and Bacillus spec.

7. The use according to claim 5, wherein the at least one pathogenic taxa comprises Staphylococcus aureus, Staphylococcus epidermidis, Pseudomonas aeruginosa, Enterococcus faecalis/faecium, E. coli, Klebsiella pneumoniae, Candida albicans, Microsporum canis, Acinetobacter baumanii, Staphylococcus intermedius and Staphylococcus pseudointermedius.

8. The use according to claims 1 to 7, wherein the chitosan has a degree of acetylation of 15% or less, more preferably 10% or less, even more preferably 5% or less or even more preferably 2.5% or less.

9. The use according to claims 1 to 8, wherein the liquid cosmetic composition comprises one further cosmetic agent.

10. The use according to claim 9, wherein the one further cosmetic agent is selected from the group comprising urea, glycolic acid, glyoxylic acid, glycerol, pentylene glycol, lactic acid, ascorbic acid, pyroglutamic acid, citric acid, tartaric acid, fumaric acid, succinic acid, malic acid, mandelic acid, aloe vera, rosa gallica, hyaluronic acid, salicylic acid, gallic acid, cellulose and derivatives thereof, pectin and derivatives thereof, gummi arabicum, dextrines, cyclodextrines, xanthan gum, thiocyanate, amino acids, sorbic acid, sodium chloride or combinations thereof.

11. The use according to claim 9 or 10, wherein the at least one further cosmetic agent comprises glycolic acid and/or lactic acid.

12. The use according to claims 9 to 11, wherein the composition comprises
0.010 to 4.0% (w/w) chitosan,
0.005 to 2.5% (w/w) glycolic acid,
0.005 to 2.5% (w/w) lactic acid,
based on the total weight of the liquid cosmetic composition.

13. The use according to claims 1 to 12, wherein the liquid cosmetic composition comprises a disinfectant.

14. The use according to claim 13, wherein the disinfectant comprises an alcohol, aldehyde, iodine, chlorine, quarternary ammonia compound, peroxide, amphotenside, phenol, alkylamine, acid and/or base.

15. The use according to claims 1 to 14, wherein the ectodermal tissue is the skin.

16. The use according to claim 15, wherein the ectodermal tissue is the epidermis.

17. The use according to claim 16, wherein the epidermis is damaged epidermis, wherein the damage comprises sunburn, acne, cuts, abrasions, cuts, pimples, blisters, stings, burns, ageing, irritated skin, radiated skin, laser treated skin, plasma treated skin, a tattoo, the removal of a tattoo, skin peeling, scar tissue, dry skin, fatty skin, cracks, stretch marks or wrinkling.

18. The use according to claim 16, wherein the epidermis is stressed epidermis, wherein the stress is caused by prostheses, endoprostheses, orthoses, exoskeletons, plasters, compression bandages, stockings, bandages, latex protection, massagers, masks for ventilation, apnea prevention, work- or protective clothing, gloves, pacifiers, tight clothes or shoes, disinfectants, cosmetic treatments, cosmetic products, preservatives, cleaning agents, sweat, lack of body hygiene, long lying or sitting, wound dressings, sunburns, burns, stings, radiation, laser treatment, plasma treatment, tattooing and/or removal of tattoos.

19. The use according to claim 18, wherein the stress is caused by prostheses, endoprostheses, orthoses, exoskeletons, plasters, compression bandages, stockings, bandages, latex protection, massagers, masks for ventilation, work- or protective clothing, gloves, pacifiers, tight clothes or shoes, long lying or sitting or wound dressings.

20. The use according to claims 1 to 19, wherein the subject's ectodermal tissue, skin, epidermis, damaged or stressed epidermis has previously been sterilized or disinfected.

21. A liquid cosmetic composition comprising
a) chitosan or a salt thereof, wherein the degree of acetylation of the chitosan is 20% or less, and
b) at least one further cosmetic agent;
wherein the pH of the liquid cosmetic composition is from about 4.0 to about 6.5;
and wherein the at least one further cosmetic agent comprises glycolic and lactic acid.

22. The liquid cosmetic composition according to claim 21, wherein the degree of acetylation of the chitosan is 15% or less, more preferably 10% or less, even more preferably 5% or less, or even more preferably 2.5% or less.

23. The liquid cosmetic composition according to claim 20 or 21, wherein the composition comprises
0.010 to 4.0% (w/w) chitosan,
0.005 to 2.5% (w/w) glycolic acid,
0.005 to 2.5% (w/w) lactic acid,
based on the total weight of the liquid cosmetic composition.

24. Kit comprising a liquid cosmetic composition according to any of 21 to 23, further comprising a disinfectant, preferentially selected from the group comprising an alcohol, aldehyde, iodine, chlorine, quarternary ammonia compound, peroxide, amphotenside, phenol, alkylamine, acid and/or base.
